(19) United States
(12) Patent Application Publication
(10) Pub. No.: US 2022/0330931 A1
(43) Pub. Date: Oct. 20, 2022

(54) FORMULATION AND PACKAGING OF COMPOSITIONS

(71) Applicant: RevBio, Inc., Lowell, MA (US)

(72) Inventors: Brian J. Hess, Charlestown, MA (US); George W. Kay, Sharon, MA (US); Michael C. Brown, Harvard, MA (US); David J. Kosh, Norton, MA (US); Andrey Marchenko, Acton, MA (US)

(21) Appl. No.: 17/639,221

(22) PCT Filed: Aug. 28, 2020
(86) PCT No.: PCT/US2020/048584

§ 371 Date: Feb. 28, 2022

Related U.S. Application Data
(60) Provisional application No. 62/899,344, filed on Sep. 12, 2019.
(60) Provisional application No. 62/894,272, filed on Aug. 30, 2019.

(51) Int. Cl. A61B 17/00; A61L 24/00; A61L 24/02

(52) U.S. Cl. CPC A61B 17/00491; A61L 24/0015; A61L 24/02; A61C 5/64

(57) ABSTRACT

The present disclosure features a composition (e.g., adhesive composition), as well as devices and related means for packaging and delivery of the same. The devices and related methods may be used for activating and mixing the components of composition (e.g., adhesive composition), for example, within a packaged device without compromising sterility.

### BACKGROUND

There is a need for improved methods for the delivery of compositions for the repair of tissue, in part to provide packaging to allow for storage and mixing of separate components of such a composition while maintaining sterility.

### SUMMARY

The present disclosure features a composition (e.g., adhesive composition), as well as devices and related means for packaging and delivery of the same. In some embodiments, the composition (e.g., adhesive composition) comprises synthetic materials. The composition may be a soft tissue adhesive or a hard tissue adhesive. In some embodiments, composition is a bone graft substitute. In some embodiments, the composition is used to adhere mineralized or nonmineralized tissue. In some embodiments, the composition is a cement.

In another aspect, the present disclosure features equipment and methods for activating and mixing the components of composition (e.g., adhesive composition), for example, within a packaged device without compromising sterility. In some embodiments, the devices and methods described herein provide the user a means for observing the mixing of multiple components of the composition (e.g., adhesive composition), as well as a way to dispense the prepared composition into the surgical field without compromising asepsis. The embodiments described herein may be used singularly or in a plurality. In some embodiments, the devices disclosed herein may be sold in packs of six. The device, compositions, and methods described herein may be used in human or nonhuman patients.

Generally, compositions, such as adhesive compositions, are prepared in the open air by combining several components in an aqueous medium. After mixing, the prepared compositions are then loaded into a specific tool for administration to a target site. These current methods require the user to perform all required steps quickly, in order to prevent premature hardening of the composition prior to placement at the desired site. There is also no reliable means for ensuring the components of the composition are thoroughly mixed in a sterile manner. In contrast, the devices and related packaging described herein provide a method for direct mixing of the components of the composition described herein in a sterile environment. For example, when ready for use, the device may be activated manually, then optionally placed in an agitator, e.g., a triturator, for mixing, all while remaining in a package designed to limit the ingress and egress of a contaminating element. In some embodiments, the agitator contacts the package via a pair of forks (e.g., custom-assembled forks) to maintain sterility of the device and composition within.

The present disclosure features a package comprising a device for storing, mixing, and administering a composition (e.g., adhesive composition), wherein the device comprises: a chamber within a main body; a barrier; a plunger; a nozzle; and a composition (e.g., adhesive composition); and wherein the package limits ingress of a contaminating element. In some embodiments, the package comprises an interior environment for the device. In some embodiments, the package comprises a clear or translucent region on at least one side of the package. In some embodiments, the package comprises at least two sheets or layers of material (e.g., a first sheet or layer of material and a second sheet or layer of material). In one embodiment, the at least two sheets or layers of material are flexible. In another embodiment, the at least two sheets or layers of material are rigid. In some embodiments, the at least two sheets or layers of material are sealed around the device.

In some embodiments, the device comprises a first chamber and a second chamber. In an embodiment, components of composition (e.g., adhesive composition) are provided as dry components (e.g., a powder or granules) within the first chamber. The dry components of the composition comprise a multivalent metal compound and an acidic compound. In some embodiments, the second chamber of the device comprises an aqueous medium (e.g., water). In some embodiments, the barrier separates a first chamber from a second chamber. In further embodiments, the barrier separates a main chamber from the nozzle. Specifically, the barrier comprises a material selected from a polymer or a metal. More specifically, the barrier has a diameter/thickness of less than 0.05 mm, e.g., 0.01 mm. In some embodiments, the contents of device are combined to form a composition (e.g., adhesive composition). More specifically, the contents of the first chamber and the contents of the second chamber are combined within the packaging to form a composition (e.g., adhesive composition). In some embodiments, the composition is comprised of a biomaterial. In some embodiments, the composition is comprised of synthetic materials. In some embodiments, the composition is a soft tissue adhesive or sealant.

In some embodiments, the composition is a hard tissue adhesive. In some embodiments, the composition is a bone graft substitute. The composition (e.g., adhesive composition) may be used to adhere mineralized or nonmineralized tissue. In some embodiments, the composition is a cement. More specifically, said biomaterial may be comprised of mineral-based compounds, organic-based compounds, or a combination thereof In some embodiments, the biomaterial may comprise hydrogels, cements, or adhesives.

The present disclosure also features a method of mixing the components of the multiple chambers, wherein the package comprising the device is agitated to mix the components of the composition (e.g., adhesive composition). In some embodiments, the package comprising the device is placed into an agitator (e.g., a triturator) for agitation. In an embodiment, the agitator (e.g., a triturator) comprises a device holder assembly (e.g., a fork assembly) comprising two arms. More specifically, the fork assembly is custom assembled to hold the package. In some embodiments, the agitator and device holder assembly, e.g., a fork assembly, maintain sterile contact with the device so as not to compromise asepsis within the device.

The present disclosure also features a method of preparing a composition (e.g., adhesive composition) comprising (a) disposing components of the composition (e.g., adhesive composition) into a device comprising: (i) a chamber within a main body; (ii) a barrier; (iii) a plunger; and (iv) a nozzle; (b) applying pressure/depressing the plunger of the device to breach the barrier, allowing the components of the composition (e.g., adhesive composition) to come into contact with each other; and (c) placing the device into an agitator, e.g., a triturator, to agitate the contents of the device; thereby preparing the composition (e.g., adhesive composition).

Additional features of the devices, packages, compositions, agitators, and methods of use thereof are described herein in the Detailed Description, Examples, Figures, and Claims.

### BRIEF DESCRIPTION OF THE DRAWINGS

FIGS. 1A-1B are depictions of an exemplary device, e.g., as described herein, contained within a package.

FIG. 2 is an image of an exemplary assembly (e.g., a device holder assembly) that may be used to clamp the device for agitation.

FIG. 3 is an image of an exemplary device contained within a package loaded into a device holder assembly for agitation.

FIG. 4 is an image depicting removal of an exemplary packaged device from a device holder assembly.

FIG. 5 is an image showing a method for opening the package to retrieve the device, e.g., by manually peeling a chevron seal.

FIG. 6 is an image depicting liberation of the device from the packaging, e.g., for dropping into a sterile field.

FIGS. 7A-7B are images of current device embodiments loaded into a device holder assembly for agitation.

FIGS. 8A-8B are images of exemplary sterile packaging applications to a model patient interface.

FIG. 9 is an image of an exemplary device undergoing sterile field transfer.

FIGS. 10A-10B are images of exemplary sterile packaging embodiments and method of agitating such embodiments.

### DETAILED DESCRIPTION

The present disclosure features compositions (e.g., adhesive compositions), as well as devices and related means for packaging and delivery of the same. Components of devices and packaging, along with the compositions (e.g., adhesive compositions), agitators, and related methods of use will be described herein in greater detail.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein.

“Alkyl” refers to a radical of a straight-chain or branched saturated hydrocarbon group. In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl (C₁) and propyl (C₃). Alkyl groups disclosed herein may be substituted or unsubstituted.

As used herein, “alkylene,” refers to a divalent radical of an alkyl group. When a range or number of carbons is provided for a particular alkylene group, it is understood that the range or number refers to the range or number of carbons in the linear carbon divalent chain.

The term “heteroalkyl,” as used herein, refers to an alkyl group, as defined herein, which further comprises 1 or more (e.g., 1 or 2) heteroatoms (e.g., non-ionizable heteroatoms) within the parent chain, wherein the one or more heteroatoms is inserted between adjacent carbon atoms within the parent carbon chain and/or one or more heteroatoms is inserted between a carbon atom and the parent molecule, i.e., between the point of attachment. Exemplary heteroatoms within a heteroalkyl group include oxygen, phosphorus, nitrogen, and sulfur atoms.

As used herein, “alkylene,” and “heteroalkylene,” refer to a divalent radical of an alkyl and heteroalkyl group respectively. When a range or number of carbons is provided for a particular alkylene or heteroalkylene group, it is understood that the range or number refers to the range or number of carbons in the linear carbon divalent chain.

As used herein, “carboxyl” refers to —C(O)OH.

As used herein, “phosphonyl” refers to —P(O)(OH)₂.

As used herein, “aryl” refers to a functional group or substituent derived from an aromatic ring containing carbon and hydrogen atoms. In some embodiments, an aryl may be derived from an aromatic hydrocarbon. Exemplary aryl groups include, but are not limited to, phenyl and naphthyl.

As used herein, “heteroaryl” refers to a functional group or substituent derived from an aromatic ring that contains carbon atoms and at least one heteroatom, such as a nitrogen, sulfur, oxygen, or phosphorus atom. Exemplary heteroaryl groups include, but are not limited to, thienyl, pyridyl, pyrimidinyl, imidazolyl, pyrrolyl, indolyl, and furanyl. Heteroaryl groups disclosed herein may be substituted or unsubstituted.

As used herein, “hydroxy” refers to —OH.

As used herein “thiol” refers to a sulfur analog of an alcohol. In some embodiments, a thiol group may include an organosulfur compound, for example, one that contains a carbon-bonded sulfhydryl. Exemplary thiol groups include —SH, —C—SH, and R—SH, where R represents an organic substituent, e.g. an aryl or alkyl.

As used herein “amino” refers to a compound that contains a nitrogen atom, for example, with a lone pair, attached to another substituent (e.g., a hydrogen atom, alkyl group, or aryl group). In some embodiments, the amino may be derived from ammonia, for example, wherein one or more hydrogen atoms have been replaced by a substituent, for example an aryl or alkyl. The amino may be organic or inorganic. In some embodiments, an amino includes —NH₂, an amino acid, a biogenic amine, trimethylamine, and aniline.

### Devices

The present invention features, in part, a device for storing, mixing, and administering a composition (e.g., an adhesive composition). The device may be comprised of several components that are assembled to store, mix, and administer a resulting prepared implantable composition, e.g., bone adhesive or cement.

The present invention disclosure provides a device comprising: a main body; a plunger; at least one barrier; and optionally a nozzle. In an embodiment, the device further comprises a sachet. In some embodiments, when fully assembled, the main body of the device has one chamber comprising the components of a composition (e.g., an adhesive composition). In some embodiments, when fully assembled, the device has more than one chamber (e.g., two chambers, three chambers) separately comprising the components of the composition. In some embodiments, when fully assembled, the device has two chambers separately comprising the components of the composition, e.g., a first chamber and a second chamber.

The device may comprise a chamber in which the components of the composition are stored and/or mixed. This chamber can be swept by a plunger that empties the chamber by expressing the chamber contents out through an orifice of the main body to an application site (e.g., bone) or optionally into a nozzle, through which the resulting implantable composition flows to an application site (e.g., bone). In some embodiments, the device comprises two chambers for storing the components of the composition. A barrier comprising the separating wall of the two chambers separating the components of the composition (e.g., the solid components and liquid components) may be breached during activation of the device prior to mixing of the said components. In an embodiment, the device comprises a plurality of barriers. In some embodiments, a second barrier separating the chamber from the nozzle is breached prior to expression of the resulting prepared composition (e.g., an adhesive composition), from the device during application.

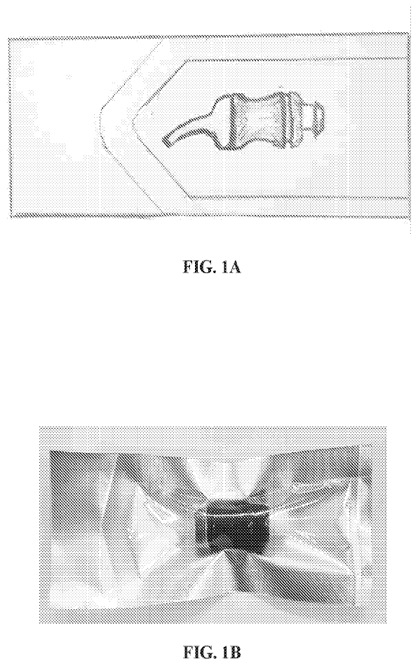

The present disclosure features a first chamber and a second chamber within the device. In some embodiments, the first chamber may include a space in the main body wherein the sachet is assembled into the proximal end of the main body and a membrane is attached at the terminal end of the main body. In other embodiments, the sachet is assembled into a space or second chamber outside the main body of the device. In some embodiments, the second chamber may include a space in the main body wherein the plunger is inserted into the proximal end of the sachet. Optionally, a membrane is attached at the terminal end of the sachet. In some embodiments, the device may further comprise a barrier separating the first chamber and the second chamber, which may be compromised through manual depression of the plunger. In further embodiments, the device may comprise a nozzle attached to the terminal end of the main body through which the prepared implantable composition, e.g., bone adhesive or cement, may be delivered. An exemplary depiction of the device disposed within a package (e.g., a package described herein) is shown in FIGS. 1A-B.

In some embodiments, the components of the composition (e.g., an adhesive composition) such as the dry components and an aqueous medium, are stored within a chamber of the device. In a preferred embodiment, the dry components of the composition (e.g., an adhesive composition), e.g., the multivalent metal compound or the acidic compound, are stored within a first chamber of the device. In a further embodiment, the aqueous medium is stored within the sachet or within a second chamber of the device. In some embodiments, the dry components of the composition (e.g., an adhesive composition) are dissolved or suspended within an aqueous medium that is stored within a chamber of the device, for example, the first chamber or the second chamber.

In some embodiments, a chamber of the device has an inner diameter less than about 50 mm. For example, a chamber of the device may have a diameter less than about 30 mm, 20 mm, or 10 mm. In some embodiments, a chamber of the device has a diameter less than about 10 mm, e.g., less than 7.5 mm, 5 mm, or 2 mm. In some embodiments, a chamber of the device has a diameter between 25 mm and 1 mm, e.g., between 15 mm and 5 mm, between 10 mm and 5 mm, or between 5 mm and 1 mm.

In some embodiments, wherein the device has more than one chamber, the first chamber has an inner diameter less than about 50 mm. For example, the first chamber may have a diameter less than about 30 mm, 20 mm, or 10 mm. In an embodiment, the first chamber has a diameter less than about 10 mm, e.g., less than 7.5 mm, 5 mm, or 2 mm. In an embodiment, the first chamber has a diameter between 25 mm and 1 mm, e.g., between 15 mm and 5 mm, between 10 mm and 5 mm, or between 5 mm and 1 mm. In some embodiments, the second chamber has an inner diameter less than about 50 mm. For example, the second chamber may have a diameter less than about 30 mm, 20 mm, or 10 mm. In some embodiments, the second chamber has a diameter less than about 10 mm, e.g., less than 7.5 mm, 5 mm, or 2 mm. In some embodiments, the second chamber has a diameter between 25 mm and 2 mm, e.g., between 15 mm and 5 mm, between 10 mm and 5 mm, or between 5 mm and 1 mm.

In some embodiments wherein the device comprises one chamber, said chamber is less than about 500 mm in length. For example, a chamber of the device may be less than about 250 mm, 100 mm, 50 mm, or 25 mm in length. In some embodiments, a chamber of the device is less than about 20 mm in length, e.g., less than 15 mm, less than 10 mm in length. In some embodiments, a chamber of the device is between 250 mm and 2 mm in length, e.g., between 100 mm and 25 mm in length, between 50 mm and 10 mm in length, or between 5 mm and 2 mm in length.

Wherein the present disclosure features a device comprising a first and second chamber, the first chamber may be less than about 500 mm in length. For example, the first chamber may be less than about 250 mm, 100 mm, 50 mm, or 25 mm in length. In some embodiments, the first chamber is less than about 20 mm in length, e.g., less than 15 mm, less than 10 mm in length. In some embodiments, the first chamber is between 250 mm and 2 mm in length, e.g., between 100 mm and 25 mm in length, between 50 mm and 10 mm in length, or between 5 mm and 2 mm in length. In some embodiments, the second chamber may be less than about 100 mm in length. For example, the second chamber may be less than about 50 mm, 25 mm, or 10 mm in length. In some embodiments, the second chamber is less than about 10 mm in length, e.g., less than 7.5 mm, 5 mm, or 2 mm in length. In some embodiments, the second chamber is between 50 mm and 2 mm in length, e.g., between 25 mm and 10 mm in length, between 10 mm and 5 mm in length, or between 5 mm and 2 mm in length.

The present disclosure also features a barrier in the device (e.g., a barrier separating the first chamber and the second chamber) as a separate component. In some embodiments, the barrier is a feature (e.g., a thin wall) integral to the sachet. In some embodiments, the barrier as a separate component may be joined or bonded to the distal end of the sachet to ensure a seal. More specifically, said seal may be created using an adhesive or by welding the barrier to the first or second chamber at the interface between the materials. Said barrier acts as the adjoining wall between a first and second chamber of the device. A primary function of the barrier may be to prevent the components of the adhesive composition or cement from prematurely mixing during the packaged state, such as when the product is in transport or on the shelf awaiting its intended use (e.g., in the surgical setting). In some embodiments, the barrier is designed to allow it to be compromised (e.g., breached) at the point of intended use (e.g., in the surgical setting). More specifically, the barrier may be compromised via puncturing or depression of a plunger.

The present disclosure features a sachet disclosed within the main body of the device. In some embodiments, the sachet may be rigid (e.g., a cup) or flexible (e.g., a pouch). The sachet may comprise materials to limit liquid or moisture egress that is contained from within the sachet during its storage and transit. After assembly of the liquid, the interference fit of other parts (e.g., plunger) or the seal (if self-contained) is such to limit liquid or moisture egress that is contained from within the sachet during its storage and transit.

In some embodiments, the barrier within the device (e.g., a barrier separating the first chamber and the second chamber) may comprise a material selected from a polymer or a metal. For example, the barrier may comprise a polyolefin, polyamide, polyester, polyaryl, polycarbonate, or a mixture or co-extrusion thereof. In some embodiments, the polymer is selected from polyvinyl chloride, polyvinylidene chloride, polyethylene, polyethylene terephthalate, polyethylene naphthalate, polystyrene, ethylene vinyl alcohol, or a mixture or co-extrusion thereof. In some embodiments, the barrier may comprise a metal selected from aluminum or tin, or a foil thereof. In some embodiments, the barrier comprises a laminate. In some embodiments, the barrier may contain a single sheet or layer of material, or may comprise a plurality of sheets or material, e.g., at least two sheets or layers of material, or at least three sheets or layers of material. In some embodiments, the barrier comprises a sheet or layer of a metal (e.g., an aluminum foil) and a sheet or layer of a polymer (e.g., polyvinyl chloride or polyvinylidene chloride).

The barrier within the device may be less than 5 mm thick. For example, the barrier may be less than about 4 mm, 3 mm, 2 mm, 1 mm, 0.75 mm, 0.5 mm, 0.25 mm, or 0.1 mm thick. In some embodiments, the barrier is less than about 0.1 mm thick, e.g., less than 0.075 mm, 0.05 mm, 0.025 mm, 0.01 mm, or 0.0075 mm thick. In some embodiments, the barrier is between 5 mm and 0.001 mm thick, e.g., between 3 mm and 0.005 mm thick, 2 mm and 0.005 mm thick, or 1 mm and 0.001 mm thick.

In embodiments in which the device comprises more than one chamber, the barrier separating the first chamber and the second chamber may be less than 5 mm thick. For example, the barrier may be less than about 4 mm, 3 mm, 2 mm, 1 mm, 0.75 mm, 0.5 mm, 0.25 mm, or 0.1 mm thick. In some embodiments, the barrier is less than about 0.1 mm thick, e.g., less than 0.075 mm, 0.05 mm, 0.025 mm, 0.01 mm, or 0.0075 mm thick. In some embodiments, the barrier is between 5 mm and 0.001 mm thick, e.g., between 3 mm and 0.005 mm thick, 2 mm and 0.005 mm thick, or 1 mm and 0.001 mm thick.

The barrier separating the first chamber and the second chamber may be less than 50 mm in diameter, for example, extending from the top to the bottom of the interior cavity of the main body of the device. In some embodiments, the barrier may be less than about 30 mm, 20 mm, 15 mm, 10 mm, 5 mm, 3 mm, 2 mm, 1 mm, 0.5 mm, or 0.25 mm in diameter. In some embodiments, the barrier is between 50 mm and 0.25 mm in diameter, e.g., between 30 mm and 5 mm, 10 mm and 2 mm, 5 mm and 1 mm, or between 2 mm and 0.25 mm in diameter.

The device may further features a plunger disposed within the sachet or main body of the device. The diameter of the plunger may be selected such that it fits within the sachet or within the main body of the device. More specifically, the diameter of the plunger and sachet or main body can have similar diameters (e.g., tolerance fit), while still allowing the plunger to advance within the sachet or main body of the device. In some embodiments, the sachet or main body has a feature to retain the plunger in a pre-set position when assembled. More specifically, this pre-set position may define the space of a chamber in the device, such as the second chamber. In some embodiments, the plunger has a feature to retain it within the sachet or main body at a pre-set position when assembled. In some embodiments, this pre-set position defines the space of the second chamber. In some embodiments, this retainment feature is overcome by sufficient force (e.g., at least 5 N, in some cases at least 50 N) to advance the plunger towards the distal end of the device.

The sachet or main body may have a feature to allow air to escape during assembly. In some embodiments, the sachet or main body has a feature to allow air to escape during use of the device when the plunger is fully advanced within the sachet. Alternatively, the plunger may have a feature to allow air to escape during assembly or during its use when the plunger is fully advanced within the sachet or main body. More specifically, this air escapement feature may be small enough such that the composition (e.g., adhesive composition) or the components of the composition cannot leak through this passage. The plunger presently disclosed may traverse the housing of the sachet until it is fully seated in the sachet housing in order to breach a barrier in the device (e.g., the barrier between the first and second chamber) and transfer the composition component(s) from one chamber to another (e.g., the first chamber to the second chamber). In some embodiments, this distance may make the proximal end of the plunger partially or completely coincident or in contact with the proximal end of the main body of the device. In some embodiments, the proximal end of the plunger is convex. In some embodiments, the proximal end of the plunger matches the shape of the base of the nozzle. In some embodiments, the plunger and sachet may traverse fully through the housing of the main body until they are fully seated towards the terminal end of the main body in the sachet housing in order to breach a barrier in the device (e.g., the barrier between the first chamber and nozzle) and transfer the composition from a chamber to the nozzle (e.g., the first chamber through the nozzle) to the intended site of application (e.g., in or onto bone).

In embodiments in which the device comprises more than one chamber, the first chamber may have an inner diameter less than about 50 mm. For example, the first chamber may have a diameter less than about 30 mm, 20 mm, or 10 mm. In some embodiments, the first chamber has a diameter less than about 10 mm, e.g., less than 7.5 mm, 5 mm, or 2 mm. In some embodiments, the first chamber has a diameter between 25 mm and 2 mm, e.g., between 15 mm and 5 mm, or between 10 mm and 5 mm.

In embodiments in which the device comprises more than one chamber, the second chamber may have an inner diameter less than about 50 mm. For example, the second chamber may have a diameter less than about 30 mm, 20 mm, or 10 mm. In some embodiments, the second chamber has a diameter less than about 10 mm, e.g., less than 7.5 mm, 5 mm, or 2 mm. In some embodiments, the second chamber has a diameter between 25 mm and 2 mm, e.g., between 15 mm and 5 mm, or between 10 mm and 5 mm.

The sachet may be less than about 100 mm in length. For example, the sachet may be less than about 50 mm, 25 mm, or 10 mm in length. In some embodiments, the sachet is less than about 10 mm in length, e.g., less than 7.5 mm, 5 mm, or 2 mm in length. In some embodiments, the sachet is between 50 mm and 2 mm in length, e.g., between 25 mm and 10 mm in length, or between 10 mm and 2 mm in length.

The plunger may be less than about 100 mm in length. For example, the plunger may be less than about 50 mm, 25 mm, or 10 mm in length. In some embodiments, the plunger is less than about 10 mm in length, e.g., less than 7.5 mm, 5 mm, or 2 mm in length. In some embodiments, the plunger is between 50 mm and 2 mm in length, e.g., between 25 mm and 10 mm in length, or between 10 mm and 2 mm in length.

The present disclosure features a method of mixing or moving the components of the composition, e.g., adhesive composition, utilizing said plunger. In some embodiments, in order to mix and/or move the components of the composition (e.g., adhesive composition) within a chamber of the device (e.g., the components of the first chamber and the components of the second chamber), the plunger is depressed, e.g., through manual or mechanical means. In some embodiments, the plunger comprises a shaft and a terminal end suitable for piercing a barrier. In some embodiments, the terminal end of the plunger is in the shape of a cone or dome. In some embodiments, the terminal end of the plunger is substantially flat. In some embodiments, the terminal end of the plunger may contain a sharp end (e.g., a sharp edge or point) suitable for piercing a barrier. The plunger may comprise a substantially uniform cross-section, for example, throughout the length and width of shaft.

The present invention discloses a method of use wherein, upon pressure applied by a user or other means, the plunger may move through the main body of the device, allowing the terminal end of the plunger (e.g., the sharp end, the dome end, or the flat end) to come into contact with the barrier in the device (e.g., the barrier separating the first chamber and the second chamber). The plunger then may breach the barrier, thus allowing the components of a chamber of the device (e.g., the components of the first chamber and the components of the second chamber) to come into contact with each other. In some embodiments, pressure, e.g., contact pressure between terminal end of plunger and barrier or hydrostatic pressure built up between plunger and barrier, applied from a user or other means results in breach or breaking the barrier (e.g., the barrier separating the first chamber and the second chamber) and allows mixing of the components of the composition (e.g., adhesive composition). In some embodiments, continued pressure applied to the plunger may allow the plunger to traverse the entire length of the main body of the device, thus pushing the interior components into the nozzle.

In some embodiments, the nozzle is joined to the main body of the device, e.g., at the distal end of the main body of the capsule system. In some embodiments, the nozzle may be joined by interference fit (e.g., snap on) or by screw retention. In some embodiments, the nozzle may be assembled during manufacturing prior to packaging and sterilization or it may be separately supplied and joined later in the sterile field by the end user at the point of intended use of the composition (e.g., adhesive composition). A range of nozzle shapes may be provided for selection of one shape best fitted for the particular application at the time of use. In some embodiments, the nozzle is optional or not required.

In some embodiments, the nozzle of the device is less than 100 mm long. For example, the nozzle may be less than about 50 mm, 40 mm, 30 mm, 20 mm, 10 mm, 5 mm, 3 mm, 2 mm, 1 mm long. In some embodiments, the nozzle is less than about 50 mm long, e.g., less than 30 mm, 20 mm, or 10 mm long. In some embodiments, the nozzle is between 50 mm and 10 mm long, e.g., between 30 mm and 10 mm long, or 20 mm and 10 long.

In some embodiments, the nozzle of the device comprises an inner diameter, e.g., at exit, of less than 5 mm. For example, the inner diameter of the nozzle at exit may be less than about 3 mm, 2 mm, 1 mm, 0.5 mm, or 0 .25 mm. In some embodiments, the inner diameter of the nozzle at exit is between 3 mm and 0.25 mm, e.g., between 3 mm and 2 mm, between 2 mm and 1 mm or between 1 mm and 0.25 mm.

In some embodiments, the nozzle of the device may be curved or straight. For example, the nozzle curvature may have a radius less than 100 mm long. More specifically, the nozzle curvature may have a radius less than about 75 mm, 50 mm, 25 mm, or 10 mm. In some embodiments, the nozzle curvature has a radius between 50 mm and 10 mm long, e.g., between 30 mm and 10 mm long, or 20 mm and 10 mm. In some embodiments, the nozzle curvature may have a radius less than 90 degrees. For example, the nozzle curvature may trace an arc less than about 75 degrees, 45 degrees, 25 degrees, or 10 degrees. In some embodiments, the nozzle curvature may trace an arc between 75 degrees and 25 degrees, e.g., between 40 degrees and 20 degrees.

In another embodiment, the nozzle of the device may be tapered. For example, the nozzle inner diameter may taper from less than 20 mm to greater than 0.20 mm at exit. In some embodiments, the inner diameter of the nozzle may taper from less than 10 mm to greater than 0.20 mm at exit, from less than 5 mm to greater than 0.20 mm at exit, from less than 3 mm to greater than 0.20 mm at exit, or from less than 2 mm to greater than 0.5 mm at exit.

In some embodiments, the nozzle of the device is shaped in a manner for use (e.g., direct use) in a surgical setting. For example, the nozzle of the device is shaped such that there is no need for a user to remove the contents of the device (e.g., the composition, e.g., adhesive composition) and transfer it to another device suitable for application.

The present disclosure also features a device comprising more than one chamber, wherein said device may contain a barrier separating the second chamber and the nozzle. In some embodiments, the barrier separating the second chamber and the nozzle are substantially similar (e.g., identical) to the barrier separating the first chamber and the second chamber. In some embodiments, the barrier separating the first chamber and the nozzle serves to prevent movement of a contaminating component into the device, and thus, for example, compromise the sterility of the composition (e.g., adhesive composition), or ingress of moisture which might activate the powder components.

A barrier within the device (e.g., the barrier separating the first chamber and the nozzle) may comprise a material selected from a polymer or a metal. For example, the barrier may comprise a polyolefin, polyamide, polyester, polyaryl, polycarbonate, or a mixture or co-extrusion thereof. In some embodiments, the polymer is selected from polyvinyl chloride, polyvinylidene chloride, polyethylene, polyethylene terephthalate, polyethylene naphthalate, polystyrene, ethylene vinyl alcohol, or a mixture or co-extrusion thereof. The barrier may comprise a metal selected from aluminum or tin, or a foil thereof. In some embodiments, the barrier comprises a laminate. The barrier may contain a single sheet or layer of material, or may comprise a plurality of sheets or material, e.g., at least two sheets or layers of material, or at least three sheets or layers of material. In some embodiments, the barrier comprises a sheet or layer of a metal (e.g., an aluminum foil) and a sheet or layer of a polymer (e.g., polyvinyl chloride or polyvinylidene chloride).

The barrier separating the second chamber and the nozzle may be less than 5 mm thick. For example, the barrier may be less than about 4 mm, 3 mm, 2 mm, 1 mm, 0.75 mm, 0.5 mm, 0.25 mm, or 0.1 mm thick. In some embodiments, the barrier may be less than about 0.1 mm thick, e.g., less than 0.075 mm, 0.05 mm, 0.025 mm, 0.01 mm, or 0.0075 mm thick. In some embodiments, the barrier may be between 5 mm and 0.001 mm thick, e.g., between 3 mm and 0.005 mm thick, 2 mm and 0.005 mm thick, or 1 mm and 0.001 mm thick.

The barrier separating the second chamber and the nozzle may be less than 50 mm in diameter, for example, extending across the main body of the device. For example, the barrier may be less than about 30 mm, 20 mm, 15 mm, 10 mm, 5 mm, 3 mm, 2 mm, 1 mm, 0.5 mm, or 0.25 mm in diameter. In some embodiments, the barrier is between 50 mm and 0.25 mm in diameter, e.g., between 30 mm and 5 mm, 10 mm and 2 mm, 5 mm and 1 mm, or 2 mm and 0.25 mm in diameter.

Once the barrier separating the second chamber and the nozzle is breached, in some embodiments, continued pressure applied to the plunger may dispense the prepared composition (e.g., adhesive composition) from the device to a target site. In some embodiments, the device is inserted into an applicator to assist the user in depressing the plunger to administer the adhesive composition, e.g., at a target site.

The device described herein may include, in part or in whole, any device for delivery of an adhesive composition known in the art, such as a device described in, e.g., U.S. Pat. Nos. 6,375,460; 8,443,970; 8,584,838; 8,893,925; and 9,775,690.

In another aspect, the present disclosure features a device having a capsule for mixing and applying dental cement, including a capsule body having a cylindrical shape, a longitudinal axis and a dispensing end; a mixing chamber located in the capsule body; wherein the capsule is designed and arranged to hold dental cement. In an embodiment, the mixing chamber has an opening region. A piston may be located in the capsule body and in the mixing chamber to be axially displaceable for expelling mixed dental cement through the opening region. An exit opening may be located at the dispensing end of the capsule body and in the opening region. A closure cap may be arranged to cover the opening region. A clamping device may be designed to rotatably connect the closure cap to the capsule body in the opening region to be in opposite fluid-sealing relationship. An unbent application cannula may be fixedly mounted to the closure cap at an angle of approximately 10° to 80° with respect to the longitudinal axis of the capsule body. In an embodiment, the device described herein includes any device disclosed in U.S. Pat. No. 6,375,460, which is incorporated herein by reference in its entirety.

In another aspect, the present disclosure features a device for mixing components, including a main body defining a main chamber, a liquid receptacle, and a plunger. The main chamber may have a distal end which has an end cap. The main chamber may also include a central aperture sealed by a frangible membrane. In an embodiment, the plunger causes liquid in the liquid receptacle to be pushed through an internal wall, having a weakened portion therein, into the main chamber to mix the liquid with a powder contained in the main chamber. The liquid and powder may be mixed to form a paste that is accessed by an applicator which is used to break the frangible membrane. In an embodiment, the paste is of the type used in dentistry. In an embodiment, the device described herein includes any device disclosed in U.S. Pat. No. 8,584,838, which is incorporated herein by reference in its entirety.

In another aspect, the present disclosure features a dispensing capsule having a diaphragm button, stake, and frangible membrane for a system for selectively dispersing the contents of a cup into an attached bottle. The dispensing capsule may have a cavity is disposed in the cup for consumable product defined by side walls and a base plate. Preloaded ingredients contained within the hermetically sealed cup may be discharged from the dispensing capsule into a bottle by simply depressing a button disposed on the diaphragm of the cup, thereby actuating the stake to thrust forward and apply concentrated pressure abaxially to the frangible membrane. Said pressure may pierce the center of the frangible membrane, causing it to rupture and open. Further, the diaphragm button may lock in a downward position, holding the stake into the opened frangible membrane to maintain the opening, thus permitting the contents to flow through the frangible membrane and exit the cavity of the cup. In an embodiment, the device described herein includes any device disclosed in U.S. Pat. No. 8,443,970, which is incorporated herein by reference in its entirety

In another aspect, the present disclosure features a container for mixing and dispensing materials, including a body having a main chamber, a dispensing nozzle, a liquid receptable, and a plunger. Said liquid receptacle may have a front portion arranged to break away upon pressure being applied by the plunger so that the plunger can traverse the entire length of the body. This may enable a charge of material in the main chamber to be entirely dispensed through a frangible wall or barrier into and through the nozzle. In an embodiment, the device described herein includes any device disclosed in U.S. Pat. No. 8,893,925, which is incorporated herein by reference in its entirety.

In another aspect, the present disclosure features a container for mixing and dispensing of material having a liquid receptacle and a plunger, wherein the receptacle further comprises a front portion and the plunger further comprises a forwardly projecting sharp protrusion having a shaft which terminates in a sharp outer end. When the plunger is depressed, the sharp protrusion may pierce a front portion of the liquid receptacle and the liquid in the receptacle is pushed hydraulically through the front portion into a main chamber, thus the liquid contacts material in the main chamber to form a mixture. The plunger may continue to be depressed so as to break the front portion of the liquid receptacle away and then push the material in the main chamber towards a dispensing nozzle. In an embodiment, the device described herein includes any device disclosed in U.S. Pat. No. 9,775,690, which is incorporated herein by reference in its entirety

The device embodiments described herein may include, in part or in whole, any device for delivery of a composition known in the art, such as a device described in any of the patents discussed above.

### Packaging

Described herein are packages for storage and sterilization of a device that contains a composition (e.g., adhesive composition), including methods for maintaining sterility of the composition through the storage and mixing phases.

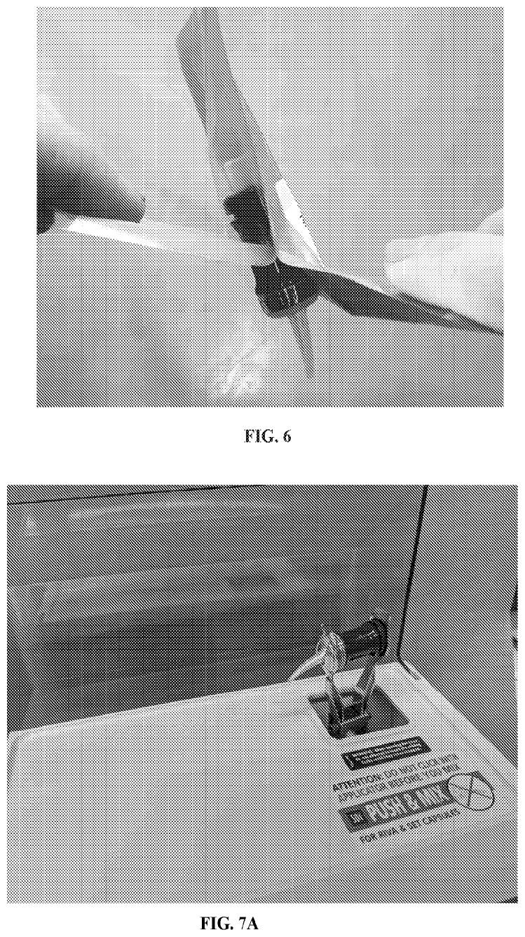

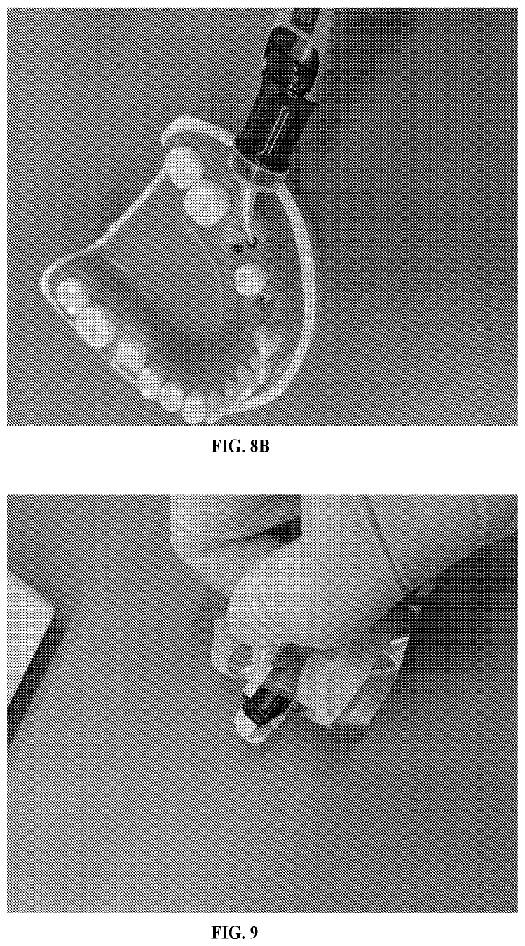

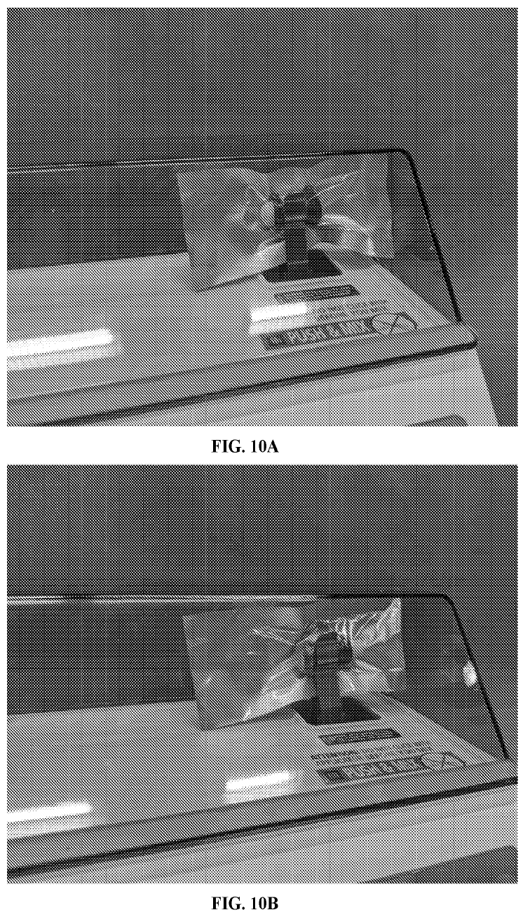

In some embodiments, the package is made from rigid or flexible components, e.g., as exemplified in FIGS. 1A-B. The flexible package may allow a user at the point of intended use to activate the composition (e.g., adhesive composition) within the device, e.g., by depressing the plunger of the device in order to breach a barrier within the device (e.g., the barrier that separates the components of the composition stored in the first chamber and the second chamber. The flexible package may allow a user at the point of intended use to advance the plunger of the device to the terminal end of the device (e.g., the second chamber) to combine the components of the composition (e.g., adhesive composition) in preparation for mixing. In some embodiments, the package may comprise a feature or surface to allow an agitator, e.g., a triturator, to positively grasp and hold the package during device activation or mixing, e.g., as exemplified in FIGS. 10A-B. In some embodiments, the package may further comprise an opening method, e.g., chevron seal, to allow for easy access to the device, as well as for aseptic retrieval, as exemplified in FIG. 6 and FIG. 9.

The package may be in the shape of a square, rectangle, or other three-dimensional shape. In some embodiments, the package comprises at least one linear dimension less than about 500 cm. For example, the package may be at less than 500 cm in length or width. In some embodiments, the package comprises at least one linear dimension less than about 500 cm, e.g., less than about 400 cm, 300 cm, 250 cm, 200 cm, 150 cm, 100 cm, 90 cm, 95 cm, 80 cm, 85 cm, 75 cm, 70 cm, 65 cm, 60 cm, 55 cm, 50 cm, 45 cm, 40 cm, 35 cm, 30 cm, 25 cm, 20 cm, 15 cm, 10 cm, 9 cm, 8 cm, 7 cm, 6 cm, 5 cm, 4 cm, 3 cm, 2 cm, 1 cm, or smaller. In another embodiment, the package comprises at least one linear dimension between about 500 cm and 1 cm, or between about 250 cm and 5 cm, or between about 100 cm and 5 cm, or between about 50 cm and 5 cm. In some embodiments, the package comprises at least two linear dimensions between about 500 cm and 1 cm, or between about 250 cm and 5 cm, or between about 100 cm and 5 cm, or between about 50 cm and 5 cm.

The package may comprise a clear or translucent region as well as an opaque region for ease of viewing or inspection of the device housed within. In some embodiments, the clear or translucent region comprises an entire aspect or side of the package. In some embodiments, the clear or translucent region comprises at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of an entire aspect or side of the package. In some embodiments, the clear or translucent region comprises between about 5% and about 95% of an entire aspect or side of the package. In some embodiments, the clear or translucent region comprises between about 10% and about 75% of an entire aspect or side of the package, or between about 10% and about 50% of an entire aspect or side of the package. In some embodiments, the entire package comprises a clear or translucent region. In some embodiments, the package does not comprise a clear or translucent region.

In some embodiments, the package may further comprise an opaque region, e.g., to shield the device from light and/or heat. In some embodiments, the opaque region comprises an entire aspect or side of the package. In some embodiments, the opaque region comprises at least 1%, 2.5%, 5%, 10%, 15%, 20%, 25%, 30%, 35%, 40%, 45%, 50%, 55%, 60%, 65%, 70%, 75%, 80%, 85%, 90%, 95%, 99%, or more of an entire aspect or side of the package. In some embodiments, the opaque region comprises between about 5% and about 95% of an entire aspect or side of the package. In some embodiments, the opaque region comprises between about 10% and about 75% of an entire aspect or side of the package, or between about 10% and about 50% of an entire aspect or side of the package. In some embodiments, the entire package comprises an opaque region. In some embodiments, the package does not comprise an opaque region.

The package may comprise a variety of materials, such as a metal or polymer. Exemplary polymers may include a polyolefin, polyamide, polyester, polyaryl, polycarbonate, or a mixture or co-extrusion thereof. In some embodiments, the polymer is selected from polyvinyl chloride, polyvinylidene chloride, polyethylene, polyethylene terephthalate, polyethylene naphthalate, polyethylene terephthalate (e.g., biaxially-oriented polyethylene terephthalate), polystyrene, ethylene vinyl alcohol, or a mixture or co-extrusion thereof. Exemplary metals include aluminum or tin, or a foil thereof. In some embodiments, the package may include a laminate, or a combination of a polymer and a metal (e.g., a metal foil).

In some embodiments, the package is comprised of at least two sheets or layers of material, wherein the device is situated between, for example, a first sheet or layer and a second sheet or layer. In some embodiments, the first sheet or layer of material comprises a clear or translucent region, and the second sheet or layer of material comprises an opaque region. Each sheet or layer of material may itself be further comprised of additional layers. For example, the first sheet or layer (e.g., on one side of the package) may be a laminate comprising several bundled sheets comprising both a polymer and a metal, as well as a clear or translucent region. In some embodiments, the first sheet or layer is a laminate comprising several bundled sheets sealed together. The sheets or layers within the package may be flexible or rigid.

In some embodiments, a first sheet or layer of material and a second sheet or layer of material are sealed around the device. The sheets or layers of the package may be sealed around device using any method known in the art, for example, heat (e.g., a heat bar seal technique). In some embodiments, a first sheet or layer of material and a second sheet or layer of material are formed around the device, e.g., allowing the space around the device for user manipulation of the device.

In some embodiments, the package may be puncture resistant, abrasion resistant, and/or resistant to tearing. In some embodiments, the package is puncture resistant, abrasion resistant, and/or resistant to tearing on all sides and dimensions, or is puncture resistant, abrasion resistant, and/or resistant to tearing on only a portion of the package.

The package may be sealed under standard atmospheric pressure (e.g., 14.7 psi at sea level). More specifically, to control contamination from contacting or corrupting the device, the package may be sealed under partial atmospheric pressure or under vacuum (e.g., <1 psi). In some embodiments, the package may be sealed in the presence of an inert gas, such as nitrogen or argon.

The package may comprise an opening mechanism to allow access to the device. In some embodiments, the opening mechanism may be of sufficient size to allow removal of the device or optionally may require the user to apply force to separate one or more sheets or layers of the packaging to remove the device, e.g., see FIG. 6. Exemplary opening mechanisms include a chevron seal. The opening mechanism may be depicted as a frangible line (e.g., a tear line), and may be located on one region (e.g. corner) of the package.

The package may comprise a grasping mechanism, i.e., a surface or feature for an agitator, e.g., a triturator, to positively grasp the package or device within during mixing. In some embodiments, the grasping mechanism is of sufficient size, roughness, or geometry to allow sufficient friction or compressive force to grasp and hold device stationary within the package during the mixing process. An exemplary grasping mechanism is a flexible pouch that conforms to the surface or geometry of the device packaged within. This may allow a user or a device holder assembly e.g., forks, clasps, or clamps, of an agitator to positively hold the device within the package during mixing. Any device grasping mechanism could work that does not compromise or cause damage to the package (e.g., tearing) or to the device during the mixing process. In some embodiments, it is useful for said grasping mechanism to limit device movement from within the package, in order for the package to not slip or eject out of the device holder assembly during the mixing process.

The present disclosure provides that the package is designed, in part, to prevent ingress and/or egress of a contaminating element. Exemplary contaminating agents include moisture (e.g., water vapor), light (e.g., ultraviolet light), oxygen, a bacterium, a fungal cell, a spore, dust, endotoxin, a pyrogen, or any other agent known for its contaminating properties. In some embodiments, the contaminating agent is a microbial agent (e.g., a bacterium, fungal cell or virus, or a spore of any of the former). In an embodiment, the contaminating agent is light. In an embodiment, the contaminating agent is moisture. In an embodiment, the contaminating agent is oxygen.

The package may provide a sterile environment for the device. In some embodiments, the device is sterilized prior to incorporation into the package. In some embodiments, the device is sterilized after incorporation into the package. The package comprising the device may be sterilized using any known sterilization technique in the art, such as autoclaving or radiation (e.g., exposure to gamma irradiation).

In some embodiments, the package may further comprise a label. The label may include information regarding the contents of the device, name of the device, date of packaging, instructions for preparing the device, or any other information required, for example, by the FDA. In some embodiments, the label is located on an opaque region of the package. In some embodiments, the label is directly printed onto the package. In some embodiments, the label is affixed onto the package.

### Agitators

The present disclosure further features a means for mixing the components of composition (e.g., adhesive composition), housed within the device, e.g., in order to activate the composition (e.g., adhesive composition). In particular, the device itself may be agitated via an agitator, e.g., a triturator, while still contained within the package, in order to reduce exposure to a contaminating agent, e.g., as exemplified in FIGS. 10A-B. In some embodiments, the mixing process takes place in a non-sterile surgical setting. In some embodiments, after breaching of the barrier, e.g., through compression of the plunger, to introduce the components of the composition (e.g., adhesive composition), from one or more chambers of the device (e.g., the second chamber to the first chamber), the package is placed into an agitator for mixing. Current agitators, such as those used in the dental field, utilize a standardized device holder assembly (e.g., a fork assembly) that can be used with many different devices (e.g., optionally enclosed within a package, as described herein). The present disclosure features embodiments in which the agitator comprises a device holder assembly, e.g., a fork assembly, comprising two device holder arms. In some embodiments, the agitator comprises a fork assembly comprising two fork arms, as exemplified in FIG. 2. In some embodiments, the device holder and fork assembly may comprise features which reduce the risk of wearing through, tearing, or otherwise disrupting the integrity of the pouch material and resulting in a breach of the sterility barrier of the pouch during loading, trituration, or removal from the agitator, e.g., a triturator.

In another aspect, the present disclosure features a mixer for dental capsules having a fork-type capsule holder including a pair of rigid arms one of which is provided with a pin that passes through an opening provided in the other arm. In an embodiment, the mixer described herein is any mixer disclosed in U.S. Pat. No. 4,890,931, which is incorporated herein by reference in its entirety.

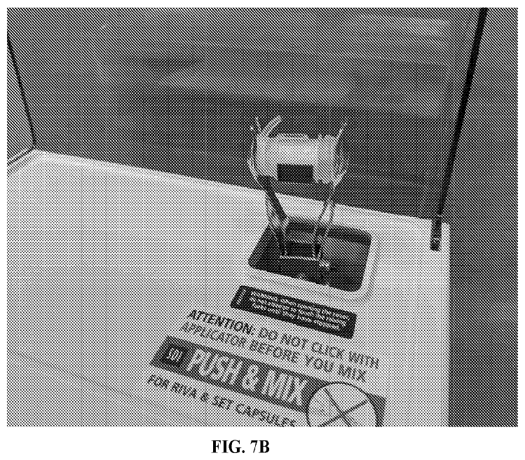

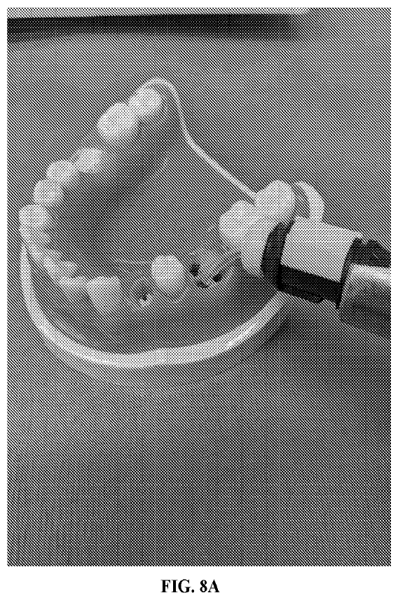

The present invention discloses a device holder assembly, e.g., fork assembly, capable of holding the device without compromising the packaging. Fork assemblies in the prior art generally are enabled to grasp a device in areas of high degrees of curvature, e.g., near the tip of the device, which can lead to deformation or tearing of the device in that section, as exemplified in FIGS. 7A-B. The fork assembly described herein may include, in part or in whole, any fork assembly embodiment known in the art. In some embodiments, the agitator comprises a custom fork assembly built to support the device without compromising the integrity of the device. In some embodiments, the two fork arms of the agitator are capable of clamping onto the body of device rather than either end of the device, as exemplified in FIGS. 10A-10B. In some embodiments, the custom fork assembly avoids contact with the dispensing tip region of the device. Wherein the custom fork arms of the agitator clamp onto the body of the device rather than each end of the device, the deliverable dose size is not limited by length of the device to be agitated. In some embodiments, such design prevents a high degree of deformation or distortion of the packaging of the device, therefore significantly reducing the risk of breaching the sterility barrier of the device. In some embodiments, the custom fork arms of the agitator are capable of avoiding puncturing the packaging of the device. In some embodiments, the custom fork assembly engages the device in a generally cylindrical region of lesser degree of curvature, providing a firm grasp which does not result in abrupt stressing or deformation of the materials comprising the packaging of the device, or barriers disposed within the device. The device (e.g., housed within the package) may be inserted into the device holder assembly (e.g., a fork assembly) between the two device holder arms. In some embodiments, the device holder arms, e.g., the fork arms, clasp the device by a compressive force generated through elastic recoil of each arm. The device holder assembly may be manufactured of materials capable of withstanding a high number of agitation cycles (e.g., hundreds of thousands of agitation cycles) without deformation, material fatigue, or unintentional release of the contents of the device and/or package containing the device.

The device may be subjected to between 5 seconds and 10 minutes of mixing in the agitating device. In some embodiments, the device is subjected to at least 2 seconds, 5 seconds, 10 seconds, 15 seconds, 20 seconds, 30 seconds, 45 seconds, 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes of mixing. In some embodiments, the device is subjected to between 2 seconds and 5 minutes of mixing, or between 5 seconds and 2.5 minutes of mixing, or between 10 seconds and 2 minutes of mixing. In some embodiments, the device may be subjected to a single round of mixing or multiple rounds of mixing.

The device (e.g., optionally enclosed within a package, as described herein) may be subjected to mixing in the agitator at a frequency of at least 1 Hz. In some embodiments, the device is subjected to mixing in the agitator at a frequency of at least 1 Hz, 10 Hz, 100 Hz, 1,000 Hz, 2,000 Hz, 3,000 Hz, 4,000 Hz, 5,000 Hz, or 10,000 Hz. In some embodiments, the device is subjected to mixing in the agitator at a frequency between 1 Hz and 10,000 Hz, or between 100 Hz and 1,000 Hz, or 500 Hz and 2,500 Hz, or 2,000 Hz and 5,000 Hz. The device may be subjected to a single round of mixing or multiple rounds of mixing.

The device may be mixed in the agitator while enclosed within the package, without causing either significant risk of compromise of sterility or rupture of the package. The device may be further mixed or shaken in the agitator while enclosed in the package without causing wrinkling or polyhedral kinking of the package. Mixing the device while still enclosed in the package may provide many benefits, including the ability for a user to accomplish mixing of the composition without comprising the sterility of the device, or also while being able to observe the device during the preparation process.

In some embodiments, once the agitation process is complete, the device may be removed from the package. In some embodiments, the device is then aseptically placed into an applicator for delivery of the composition (e.g., adhesive composition) to a target site.

### Compositions

The present disclosure features compositions (e.g., adhesive compositions) comprising a mixture of a multivalent metal salt (e.g., tetra-calcium phosphate, tricalcium phosphate or a combination thereof), a compound of a Formula (e.g., Formula (I), Formula (II), Formula (III), Formula (IV), or a combination thereof), and an aqueous medium, e.g., water. The multivalent metal salt, e.g., tetra-calcium phosphate, and a compound, e.g., Formula (I), may be comprised of a powder component. In some embodiments, the multivalent metal salt and the compound may comprise two separate powder components. In some embodiments, the powdered multivalent metal salt and the powdered compound, e.g., Formula (I), may be comprised as a singular powder component.

In some embodiments the powder component or components are formed as a loose powder. The powder component or components may be formed as dense pelletized versions. The pellets may be in the shape of a tablet. The pelletized powder components may be shaped as cylinders, e.g., approximately right cylinders or cylinders with mildly convex bases. In an embodiment, the single or plurality of cylindrical shaped pellets are loaded with the cylinder axis parallel or approximately coincident with the long axis of the dry components chamber, which itself may be approximately cylindrical in shape. The diameter of these pellets may be nearly the inner diameter of the chamber containing them. For example, wherein the pellets are located within the first chamber, the diameter may be nearly 50mm, e.g., between 30mm and 45mm. In some embodiments, a single pellet powder is utilized within the device as described herein. In some embodiments, a plurality of pellets is utilized within the device as described herein. In an embodiment, wherein multiple cylindrical pellets are used, said pellets may be stacked end-to-end. Such a stack may be space-filling or nearly space filling. The chamber may be greater in length than the single cylinder or stack of cylinders housed within it. In some embodiments, the space created therein may allow such movement of the cylinders as to generate sufficient impact energy to fragment them during trituration.

Exemplary multivalent metal salts may be organic or inorganic in nature and include calcium phosphates (e.g., hydroxyapatite, octacalcium phosphate, tetra-calcium phosphate, tricalcium phosphate), calcium nitrate, calcium citrate, calcium carbonate, magnesium phosphates, sodium silicates, lithium phosphates, titanium phosphates, strontium phosphates, barium phosphates, zinc phosphates, calcium oxide, magnesium oxide, and combinations thereof.

The amount of each multivalent metal salt (e.g., a calcium phosphate or calcium oxide or a combination thereof) may vary, e.g., between about 10% to about 90 weight by weight (w/w) of the total composition. In some embodiments, the amount of the multivalent metal salt (e.g., a calcium phosphate or calcium oxide or a combination thereof) is in the range of about 10% to about 90%, about 15% to about 85%, about 20% to about 80%, about 30% to about 75%, about 40% to about 70%, or about 50% to about 65% w/w of the total composition. In some embodiments, the amount of the metal salt (e.g., a calcium phosphate or calcium oxide or a combination thereof) is in the range of about 5% to about 95%, about 10% to about 85%, about 15% to about 75%, about 20% to about 65%, about 25% to about 55%, or about 35% to about 50% w/w of the total composition.

The multivalent metal salt may comprise an alkaline earth metal, e.g., beryllium, magnesium, barium, radium, strontium, or calcium. In some embodiments, the multivalent metal salt may comprise a mixed salt of several metal ions, e.g., a mixed salt of alkali earth metal ions. In some embodiments, the multivalent metal salt comprises calcium. In an embodiment, the multivalent metal salt comprises calcium and phosphate. In an embodiment, the multivalent metal salt comprises tetra-calcium phosphate. The composition may comprise a plurality of multivalent metal salt compounds. In some embodiments, the plurality comprises tetra-calcium phosphate and at least one other multivalent metal salt compound. In an embodiment, the multivalent metal salt comprises hydroxyapatite. In an embodiment, the multivalent metal salt comprises tricalcium phosphate. The tricalcium phosphate may comprise either alpha tricalcium phosphate or beta tricalcium phosphate. The multivalent metal salts may comprise an oxide. The multivalent metal salt may comprise calcium oxide. In some embodiments, the multivalent metal salt compound does not comprise tetra-calcium phosphate. In an embodiment, the composition comprises tricalcium phosphate and calcium oxide.

In some embodiments, the multivalent metal salt is initially provided as a powder or as a granule. These powders may exhibit a mean particle size of about 0.001 to about 1 mm, about 0.001 to about 0.25 mm, about 0.005 to about 0.15 mm, about 0.25 to about 0.75 mm, 0.25 to about 0.5 mm, 0.1 to about 0.05 mm, about 0.015 to about 0.025 mm, about 0.02 to about 0.06 mm, about 0.02 to about 0.04 mm, about 0.04 to about 0.1 mm, about 0.04 to about 0.06 mm, about 0.06 to about 0.15 mm, or about 0.06 to about 0.125 mm. In an embodiment, the powder may have a mean particle size of less than about 1 mm. In an embodiment, the particle size distribution may be multi-modal to include any combination of mean particle sizes as previously described. These granules may exhibit a mean granule size of about 0.05 mm to about 5 mm, about 0.1 to about 1.5 mm, about 0.125 to 1 mm, 0.125 to 0.5 mm, about 0.125 to 0.25 mm, about 0.25 to 0.75 mm, about 0.25 to 0.5 mm, about 0.5 to 1 mm, or about 0.5 to 0.75 mm. The granule size distribution may be multi-modal to include any combination of mean granule sizes as previously described. In an embodiment, the granules may be supplied with a various proportion of porosity and a various size of internal pores. Said pores may communicate with each other. The pores may communicate with the granule surface. In some embodiments, the pores do not communicate with each other. In some embodiments, the pores do not communicate with granule surface. In some embodiments, varying sizes of said powders or granules may be used in the adhesive composition. In some embodiments, said powders may be condensed and provided in pellet form. In some embodiments, the pelletized form of the multivalent metal salt may be provided as a pelletized powder of the multivalent metal salt and a compound of a Formula, e.g., Formula (I).

In the present disclosure, a multivalent metal salt (e.g., tetra-calcium phosphate) may react with a compound of a Formula (e.g., Formula (I), Formula (II), Formula (III), Formula (IV), or a combination thereof to form an adhesive composition when combined with an aqueous medium.

In some embodiments, the aqueous medium comprises water (e.g., sterile water), saliva, buffers (e.g., sodium phosphate, potassium phosphate, or saline (e.g., phosphate buffered saline)), blood, blood-based solutions (e.g., plasma, serum, bone marrow), spinal fluid, dental pulp, cell-based solutions (e.g, solutions comprising fibroblasts, osteoblasts, platelets, odontoblasts, stem cells (e.g., mesenchymal stem cells) histiocytes, macrophages, mast cells, or plasma cells), or combinations thereof in the form of aqueous solutions, suspensions, and colloids. In some embodiments, the aqueous medium comprises sterile water, distilled water, deionized water, sea water, or fresh water.

The aqueous medium may comprise water from the environment, e.g., fresh water, saltwater or brackish water from the oceans, seas, bays, rivers, streams, ponds or other moving or standing water sources.

In an embodiment, the aqueous medium comprises a compound, e.g., of Formula (I), in suspension in aqueous medium. This suspension of the compound of Formula (I) may be contained in a second chamber in the main body of the device.

Compositions described herein may comprise a small molecule anionic reactant and a mineral salt of a multivalent metal, for example, in an aqueous medium. The small molecule anionic reactant may comprise a compound of a Formula (e.g., Formula (I), Formula (II), Formula (III), or Formula (IV)) or combinations thereof.

In an embodiment, the compositions (e.g., adhesive compositions) disclosed herein may comprise an acidic compound of Formula (I):

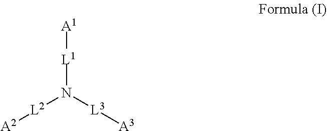

wherein: each of A¹, A², and A³ is independently selected from an acidic group (e.g., a carboxyl or phosphonyl); and each of L¹, L², and L³ is independently bond, alkylene (e.g., C₁-C₆ alkylene), or heteroalkylene (e.g., C₁-C₆ heteroalkylene).

In some embodiments, each of A¹, A², and A³ is independently a carboxyl or phosphonyl. In some embodiments, A¹ is carboxyl, and A² and A³ are phosphonyl. In some embodiments, A¹, A² and A³ are phosphonyl. In some embodiments, each of L¹, L², and L³ is C₁-C₃ alkylene. In some embodiments, each of L¹, L², and L³ is C₁ alkylene.

In some embodiments, the compound of Formula (I) is a compound of Formula (I-a) or (I-b):

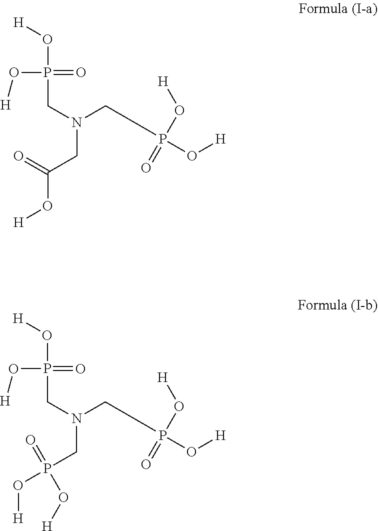 [SMILES: O=C(O)CN(CCP(=O)(O)O)CP(=O)(O)O ; O=P(O)(O)CCN(CP(=O)(O)O)CP(=O)(O)O]

In some embodiments, the aqueous medium is water. In some embodiments, the composition further comprises an additive.

In an embodiment, the compositions (e.g., adhesive compositions) disclosed herein may comprise an acidic compound of Formula (II):

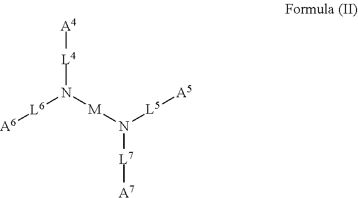

wherein each of A⁴, A⁵, and A⁶, is independently selected from an acidic group (e.g., a carboxyl or phosphonyl); A⁷ is selected from an acidic group (e.g., a carboxyl or phosphonyl), a hydrogen atom, an alkyl, an aryl, a hydroxy group, a thio group, and an amino group; each of L⁴, L⁵, L⁶, and L⁷ is independently bond, alkylene (e.g., C₁-C₆ alkylene), or heteroalkylene (e.g., C₁-C₆ heteroalkylene); and M is alkylene (e.g., C₁-C₆ alkylene) or heteroalkylene (e.g., C₁-C₆ heteroalkylene).

In some embodiments, A⁴, A⁵, A⁶ and A⁷ are carboxyl. In some embodiments, L⁴, L⁵, L⁶, and L⁷ are C₁-C₃ alkylene. In some embodiments, L⁴, L⁵, L⁶, and L⁷ are C₁ alkylene. In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups. In some embodiments, the compound of Formula (II) includes three, four, five, six, or more N-carboxymethylene groups.

In some embodiments, the compound of Formula (II) is a compound of Formula (II-a), (II-b), (II-c), (II-d), (II-e), or (II-f):

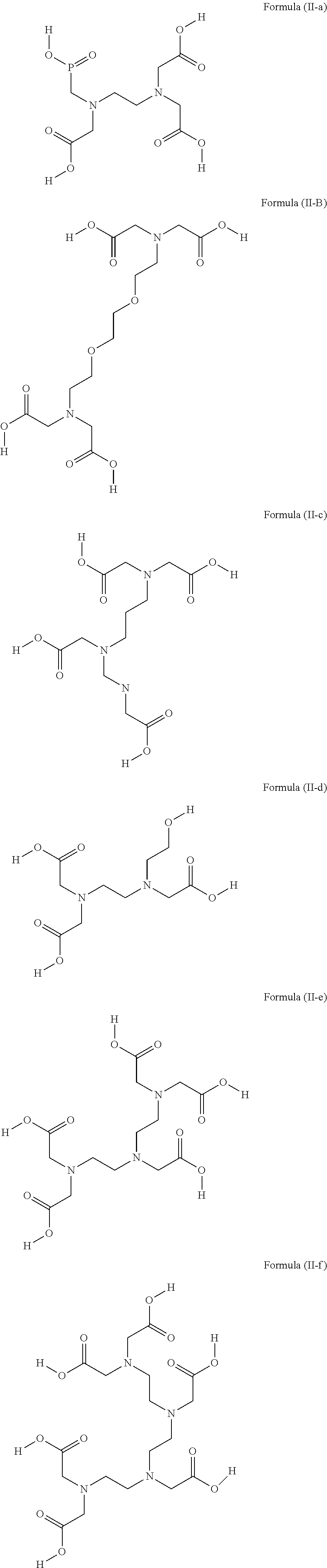

In some embodiments, the aqueous medium is water. In some embodiments, the composition further comprises an additive.

In an embodiment, the compositions (e.g., adhesive compositions) disclosed herein may comprise an acidic compound of Formula (III):

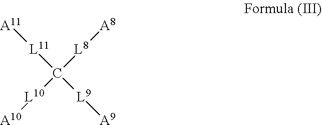

wherein each of A⁸ and A⁹ is independently selected from an acidic group (e.g., a carboxyl or phosphonyl); each of A¹⁰ and A¹¹ is independently selected from an acidic group (e.g., a carboxyl or phosphonyl), a hydrogen atom, an alkyl, aryl, a hydroxy group, a thio group, and an amino group; each of L⁸, L⁹, L¹⁰ and L¹¹ is independently bond, alkylene (e.g., C₁-C₆ alkylene), or heteroalkylene (e.g., C₁-C₆ heteroalkylene).

In some embodiments, A⁸, A⁹, and A¹⁰ are carboxyl. In some embodiments, A¹⁰, A¹¹, are a hydrogen atom. In some embodiments, A¹¹ is a hydroxy or amino group. In some embodiments, L⁸, L⁹, L¹⁰, and L¹¹ are a bond. In some embodiments, L⁸ and L⁹ are C₁-C₃ alkylene. In some embodiments L¹¹ is a heteroalkylene (e.g., C₁-C₆ heteroalkylene). In some embodiments L¹¹ is methyl enethiomethylene.

In some embodiments, the compound of Formula (III) is a compound of Formula (III-a), (III-b), (III-c), or (III-d):

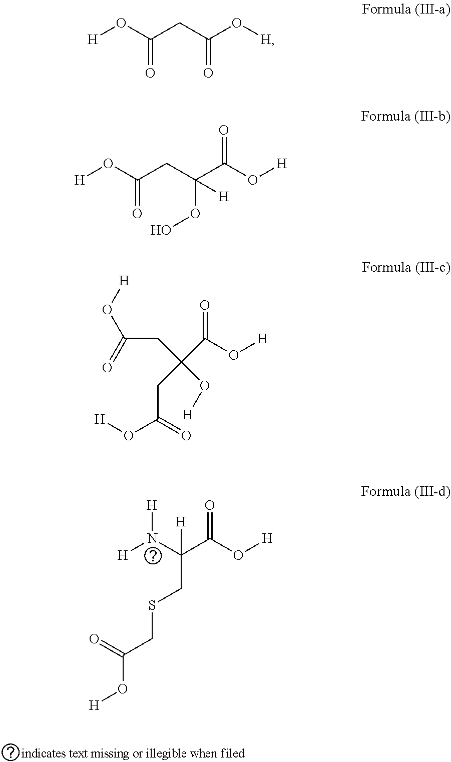

In some embodiments, the aqueous medium is water. In some embodiments, the composition further comprises an additive.

In some embodiments, the compositions (e.g., adhesive compositions) disclosed herein may comprise an acidic compound of Formula (IV):

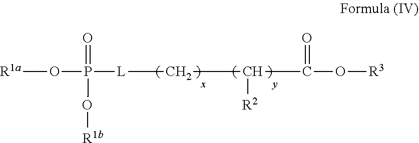

wherein: L is O, S, NH, or CH₂; each of R^{1a} and R^{1b} is independently H, an optionally substituted alkyl, or an optionally substituted aryl; R² is H, NR^{4a}R^{4b}, C(O)R⁵, or C(O)OR⁵; R³ is H, an optionally substituted alkyl, or an optionally substituted aryl; each of R^{4a} and R^{4b} is independently H, C(O)R⁶, or an optionally substituted alkyl; R⁵ is H, an optionally substituted alkyl, or an optionally substituted aryl; R⁶ is an optionally substituted alkyl or an optionally substituted aryl; and each of x and y is independently 0, 1, 2, or 3.

In some embodiments, L is O or S. In some embodiments, L is O. In some embodiments, each of R^{1a} and R^{1b} is independently H. In some embodiments, L is O, and each of R^{1a} and R^{1b} is H. In some embodiments, R² is selected from H, NR^{4a}R^{4b}, and C(O)R⁵. In some embodiments, R² in NR^{4a}R^{4b}. In some embodiments, R² is NR^{4a}R^{4b} and each of R^{4a} and R^{4b} is independently H. In some embodiments, L is O, each of R^{1a} and R^{1b} is independently H, R² is NR^{4a}R^{4b}, and each of R^{4a} and R^{4b} is independently H. In some embodiments, R³ is H. In some embodiments, L is O, each of R^{1a} and R^{1b} is independently H, R² is NR^{4a}R^{4b}, each of R^{4a} and R^{4b} is independently H, and R³ is H. In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine. In some embodiments, the aqueous medium is water.

In some embodiments, the amount of compound of a Formula (i.e., Formula (I), Formula (II), Formula (III), or Formula (IV), or a combination thereof) may vary, e.g., between about 10% to about 90% weight by weight (w/w) of the total composition. In some embodiments, the amount of compound of a Formula (i.e., Formula (I), Formula (II), Formula (III), or Formula (IV), or a combination thereof) is in the range of about 10% to about 90%, about 15% to about 85%, about 20% to about 80%, about 30% to about 75%, about 40% to about 70%, or about 50% to about 65% w/w of the total composition. In some embodiments, the amount of compound of a Formula (i.e., Formula (I), Formula (II), Formula (III), or Formula (IV), or a combination thereof) is in the range of about 5% to about 95%, about 10% to about 85%, about 15% to about 75%, about 20% to about 65%, about 25% to about 55%, or about 35% to about 50% w/w of the total composition.

The compound may be initially provided as a powder or as a granule. These powders may exhibit a mean particle size of about 0.001 to about 1 mm, about 0.001 to about 0.25 mm, about 0.005 to about 0.15 mm, about 0.25 to about 0.75 mm, 0.25 to about 0.5 mm, 0.1 to about 0.05 mm, about 0.015 to about 0.025 mm, about 0.02 to about 0.06 mm, about 0.02 to about 0.04 mm, about 0.04 to about 0,1 mm, about 0.04 to about 0.06 mm, about 0.06 to about 0.15 mm, or about 0.06 to about 0.125 mm. In an embodiment, the powder may have a mean particle size of less than about 1 mm. The particle size distribution may be multi-modal to include any combination of mean particle sizes as previously described. These granules may exhibit a mean granule size of about 0.05 mm to about 5 mm, about 0.1 to about 1.5 mm, about 0.125 to 1 mm, 0.125 to 0.5 mm, about 0.125 to 0.25 mm, about 0.25 to 0.75 mm, about 0.25 to 0.5 mm, about 0.5 to 1 mm, about 0.5 to 0.75 mm. In some embodiments, the granule size may be multi-modal to include any combination of mean granule sizes as previously described. The granules may be supplied with a various proportion of porosity and a various size of internal pores. The pores may communicate with granule surface or not. In some embodiments, varying sizes of said powders or granules may be used in the adhesive composition.

The compositions (e.g., adhesive compositions) may further comprise an additive. Particularly, an additive may be used to impart additional functionality to the composition of the disclosure, such as improving or affecting the handling, texture, durability, strength, or resorption rate of the material, or to provide additional cosmetic or medical properties. Exemplary additives may include salts (e.g., calcium carbonate, calcium bicarbonate, sodium carbonate, sodium bicarbonate, sodium chloride, potassium chloride), fillers, formulation bases, viscosity modifiers (e.g., polyols (e.g., glycerol, mannitol, sorbitol, trehalose, lactose, glucose, fructose, or sucrose)), abrasives (e.g., bone fragments), coloring agents (e.g., dyes, pigments, or opacifiers), flavoring agents (e.g., sweeteners), medications that act locally (e.g., anesthetics, coagulants, clotting factors, chemotactic agents, and agents inducing phenotypic change in local cells or tissues), medications that act systemically (e.g., analgesics, anticoagulants, hormones, enzyme co-factors, vitamins, pain relievers, anti-inflammatory agents, chemotactic agents, or agents inducing phenotypic change in local cells or tissues), antimicrobial agents (e.g., antibacterial, antiviral, or antifungal agents) or combinations thereof. In some embodiments, the additive comprises a polymer. The biologically active substances (e.g., medicines) in the categories above might include active substances or precursors, which become biologically active upon modification after interaction with the surrounding environment. The substances might be synthetic, semisynthetic, or biologically derived (e.g., peptides, proteins, or small molecules). The substances might include, but not be limited to anti-inflammatories (e.g., steroids, nonsteroidal anti-inflammatory drugs, cyclooxygenase inhibitors), complement proteins, bone morphogenic factors and proteins, hormones active locally or systemically (e.g., parathyroid hormone, calcitonin), or other small molecules (e.g., calciferols).

In some embodiments, the additive is a polymer. Suitable polymers incorporated as additives into the composition (e.g., adhesive composition) may contain functional groups that contain electronegative atoms as the bonding sites of the polymer surfaces to the available metal ions, such as electronegative carbonyl oxygen atom(s) of the ester group or electronegative nitrogen atom(s) of the amine group as the bonding sites of the polymer surfaces to the available metal ions. These functional groups can be either in the backbone chain of the polymer or in groups pendant to the polymer chain. These polymeric based compounds may include, but are not limited to, one or more of the following; poly(L-lactide), poly(D,L-lactide), polyglycolide, poly(caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin and/or mixtures thereof In addition, copolymers of the above homopolymers also can be used.

The general structural nature of a polymer (e.g., a polymer used as an additive in an composition (e.g., adhesive composition) described herein) may include a linear homo and copolymer, a cross linked polymer, a block polymer, a branched polymer, a hyper branched polymer, or a star shaped polymer. The polymers can be added to the formulation in the form of a solution, powder, fiber, resin, liquid crystal, hydrogel, chip, flake, granule, and the like. The polymeric material can be included directly within the composition (e.g., adhesive composition) or can be an adjunct that is applied in situ as the cement is applied to the bone.

In some embodiments, the composition comprises a plurality of said additives. Certain additives may be provided as powders or granules or solutes or any combination thereof. These powders may exhibit a mean particle size of about 0.001 to about 1 mm, about 0.001 to about 0.25 mm, about 0.005 to about 0.15 mm, about 0.2 to about 0.75 mm, 0.25 to about 0.5 mm, 0.1 to about 0.05 mm, about 0.015 to about 0.025 mm, about 0.02 to about 0.06 mm, about 0.02 to about 0.04 mm, about 0.04 to about 0.1 mm, about 0.04 to about 0.06 mm, about 0.06 to about 0.15 mm, or about 0.06 to about 0.125 mm. The powder may have a mean particle size of less than about 1 mm. The particle size distribution may be multi-modal to include any combination of mean particle sizes as previously described. These granules may exhibit a mean granule size of about 0.05 mm to about 5 mm, about 0.1 to about 1.5 mm, about 0.125 to 1 mm, 0.125 to 0.5 mm, about 0.125 to 0.25 mm, about 0.25 to 0.75 mm, about 0.250 to 0.5 mm, about 0.5 to 1 mm, or about 0.5 to 0.75 mm. The granule size distribution may be multi-modal to include any combination of mean granule sizes as previously described. The granules may be supplied with a various proportion of porosity and a various size of internal pores. The pores may communicate with granule surface. In some embodiments, the pores do not communicate with granule surface. In some embodiments, varying sizes of said powders or granules may be used in the composition (e.g., adhesive composition).

In some embodiments, certain additives may be provided as fibers. The fibers may exhibit a mean fiber diameter of about 0.01 mm to about 2 mm, about 0.01 mm to about 0.5 mm, or about 0.025 mm to about 0.075 mm. These fibers may exhibit a mean fiber length of about 0.025 mm to about 50.0 mm, about 0.5 mm to 10 mm, or about 1 mm to about 3.5 mm. The fiber diameter distribution or length distribution may be multi-modal to include any combination of mean fiber diameter or length.

The compositions (e.g., adhesive compositions) described herein may be applied to the surface of a structure in its fluid or semi-solid state by means of an injection delivery device or by application using an instrument such as a spatula or cannula. The viscosity of the adhesive composition when in its fluid state may be as low as about 100 cP to about 10,000 cP and when it reaches its semi-solid state from about 10,000 cP to about 250,000 cP. The viscosity and cohesion properties of the composition will facilitate the ability to squeeze the material through a needle or cannula as small as 18 gauge when the viscosity is in the low range of its fluid state. With viscosities in the semi-solid state, the shape and amount of material can be altered through spreading or removal techniques without substantially effecting the strength of the set material. In some embodiments, the working time of the adhesive composition is when the viscosity is between about 100 cP to about 250,000 cP.

The compositions (e.g., adhesive compositions) described herein may have a tacky state after mixing with an aqueous medium. This tacky property is retained for a number of days (e.g., up to 7 days, up to 3 days, up to 1 day), up to hours (e.g., up to 12 hours, up to 4 hours, up to 1 hour), up to minutes (e.g., up to 30 minutes, up to 12 minutes, up to about 4 minutes, up to about 2 minutes, up to about 1 minute), or seconds (e.g., up to 30 seconds, up to 5 seconds, up to 2 seconds),after mixing with the aqueous medium. The time of the tacky state may be dependent on a number of factors including relative ratio of the components, the particle sizes of the component materials, the presence of additives and the like, or the temperature of the environment. In some embodiments, said tacky state enables the compositions to adhere to surfaces, optionally without the need for external clamping or other application of pressure. In some embodiments, in the tacky state, the compositions will adhere bone to bone and bone to other materials. In some embodiments, the compositions may adhere materials such as stainless steel, titanium, zirconia, polyether ether ketone, steel, aluminum, copper, brass, aragonite, calcite, cement, alumina, concrete, ceramics, rock, glass, and other metals or substances. In some embodiments, during the tacky state the contacting surfaces may be held together by the adhesive composition itself, without the need for external force, until the composition sets to its final hardened cement state. In some embodiments, the tacky state can allow the materials to be positioned or repositioned without appreciable loss of cured strength.

The amount of force needed to remove two adherent pieces of material from each other during the tacky state is the tack strength. The compositions (e.g., adhesive compositions) described herein, when applied to join or affix two surfaces, may have a tack stress as measured by tensile or shear loads during the tacky state, from about 10 kPa to about 250 kPa and preferably from about 50 kPa to about 150 kPa. The tack stress may be sufficiently high that the items to be joined need not be held or clamped together unless there is an opposing strength of the items greater than the tack strength. During the tacky state the materials may be positioned, repositioned or re-opposed several times without appreciable loss of cured adhesive strength.

In some embodiments, the composition (e.g., adhesive composition) may exhibit an adhesive strength in the cement-like state in the range of about 100 kPa to about 12,000 kPa, depending on the application and the particular components and ratios of components in said adhesive compositions. In some embodiments, the adhesive strength of the composition (e.g., adhesive composition) in the cement-like state is between about 100 kPa and e.g., about 10,000 kPa, about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the composition (e.g., adhesive composition) in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the composition (e.g., adhesive composition) in the cement-like state is in the range of about 200 kPa and about 2,500 kPa. In some embodiments, the adhesive strength of the composition (e.g., adhesive composition) in the cement-like state is greater than 100 kPa.

In some embodiments, the compositions, e.g. adhesive compositions, adopt a pliable working or putty state after mixing with an aqueous medium prior to hardening, which is present for up to about one week or less, one day or less, one hour or less, 30 minutes or less, depending on the components of said compositions and the conditions of the application, e.g., temperature.

In some embodiments, the adhesive compositions and cements adopt a pliable working or putty state for less than or equal to about one week after mixing with an aqueous solution or suspension, e.g., less than about six days, less than about five days, less than about four days, less than about three days, less than about two days, less than about one day, less than about twelve hours, less than about one hour, less than about 30 minutes, less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

In some embodiments, during the putty state, which follows the tacky state, the adhesive compositions and cements can be shaped or sculpted, e.g., to fill voids in bone or acquire a desired contour, size or form. The combined time of the tacky state and the putty state is referred to herein as working time. In some embodiments, the adhesive compositions may have a working time of up to at least 3 minutes, up to at least 5 minutes, up to at least 8 minutes, up to at least 12 minutes, or up to at least 15 minutes from initial mixing, after which time the compositions have sufficiently begun hardening.

In some embodiments, following the putty state, the adhesive compositions and cements adopt a hard, cement-like state. This process of conversion from the pliable working state to the cement-like state may be referred to as “hardening,” “curing,” or “setting.” The present invention disclosure provides that the adhesive compositions and cements may harden, cure, or set such that the materials that have been affixed to each other with the adhesive compositions cannot be separated without the application of significant force. In some embodiments, the compositions typically will begin to harden within about 8 minutes, e.g., within about 5 minutes, within about 3 minutes, or within about 15 minutes, after mixing with the aqueous medium near room or body temperature. In some embodiments, compositions may be formulated to harden within a specific amount of time. For example, certain formulations may harden within less than 8 minutes, e.g., less than 30 seconds. In some embodiments, other formulations may harden within more than 8 minutes, for example, more than about 12 minutes, more than about 15 minutes, more than one day or about one week. The variance in hardening times may be due to the composition or the environment (e.g., temperature). In some embodiments, hardening time may range between less than 30 seconds to more than one day, under the same external conditions. The described tacky, putty, and set state occur in a wet environment or dry environment.

In some embodiments, the particular components of the composition may be selected to achieve the desired strength depending on the intended use of the composition (e.g., adhesive composition). In some embodiments, a skilled practitioner (e.g., a doctor, dentist, surgeon, nurse, medic, emergency technician, carpenter, mechanic, plumber, or other suitable person) may alter the specific components to achieve the desired properties (e.g., desired adhesive properties) of said composition or cement based on the intended use or desired outcome.

In some embodiments, the composition comprises a self-setting material (e.g., a biomaterial) that requires preparation by adding at least two components together in situ. In some embodiments, the composition does not have adhesive properties. For example, a non-adhesive composition may hold an object (e.g., a dental implant) into or on a bone but not adhere to the surface of the implant site. In some embodiments, the composition comprises a synthetic material or a naturally occurring material. The composition may comprise calcium phosphate and/or polymethyl methacrylate. In some embodiments, the composition is used to repair mineralized tissue, e.g., bone, or nonmineralized tissue, e.g., fascia, tendons or ligaments. Exemplary compositions for use with the device described herein further include NoriaN, HydroSet, Simplex, Mimix, OsStic, Palacos, and Bond Apatite.

In some embodiments, the device described herein comprises a two-component calcium phosphate cement having a liquid component and a dry component, and a preparation means for combining the two components of the cement while present in the storage means. In an embodiment, the composition must be removed from the device prior to use in a surgical setting. In an embodiment, the composition is removed from the device and transferred to a second device suitable for application at an intended implantation site. In an embodiment, the composition is NoriaN. In an embodiment, the device described herein includes any device disclosed in U.S. Pat. No. 6,149,655, which is incorporated herein by reference in its entirety.

In some embodiments, the device described herein comprises a calcium phosphate composition comprising: (1) at least two calcium phosphate minerals; (2) at least one reaction retarding agent; (3) at least one binding agent; and (4) at least one sodium phosphate, wherein one of said at least two calcium phosphate minerals contains a stabilizing agent, and wherein said calcium phosphate composition is rapid setting. In some embodiments, the device described herein comprises a putty calcium phosphate composition (e.g., a premixed composition) comprising at least two calcium phosphate minerals, at least one reaction retarding agent, at least one binding agent, at least one sodium phosphate, and at least one nonaqueous extender, wherein one of said at least two calcium phosphate minerals contains a stabilizing agent. In an embodiment, the composition is HydroSet. In an embodiment, the device described herein includes any device disclosed in U.S. Pat. Nos. 7,459,018 and 7,892,347, each of which is incorporated herein by reference in its entirety.

In some embodiments, the device described herein comprises α-TCP, a silicate compound, and a phosphorylated amino acid. In an embodiment, the composition is Ostic. In an embodiment, the device described herein includes any device disclosed in International Patent Publication WO2018060287A1, which is incorporated herein by reference in its entirety.

In some embodiments, the device described herein comprises (a) a liquid component comprising a monomer of an acrylic ester and (b) a powdered component comprising a terpolymer of methyl methacrylate, butyl methacrylate, and styrene. A prepared terpolymer, based on the weight of the powdered component, has between 55 to 89.5% methyl methacrylate, 10 to 40% butyl methacrylate and 0.5 to 5% styrene. In an embodiment, the composition is Simplex bone cement. In an embodiment, the device described herein includes any device disclosed in U.S. Pat. No. 5,276,070, which is incorporated herein by reference in its entirety.

In some embodiments, the device described herein comprises a light-curable resin mixed with a glass ionomer adhesive. The components of the composition may be prepared by mixing glass ionomer crystals with an aqueous solution. In an embodiment, the composition is Mimix. In an embodiment, the device described herein includes any device disclosed in U.S. Pat. No. 6,086,365, which is incorporated herein by reference in its entirety.

In some embodiments, the device described herein comprises a pasty PMMA dough, comprising a cement powder component and a monomer liquid. During the mixing of powder component and monomer component, the activator, N,N-dimethyl-p-toluidine may react with dibenzoylperoxide while forming radicals. The radicals thus formed trigger the radical polymerisation of the PMMA, upon which the viscosity of the cement dough increases until the cement dough solidifies. In an embodiment, the composition is Palacos. In an embodiment, the device described herein includes any device disclosed in U.S. Patent Publication No. 2018-0256233, which is incorporated herein by reference in its entirety.

In some embodiments, the device described herein comprises a bi-phasic calcium sulfate bone grafting composition. Embodiments of such are characterized by a chemical composition of cementitious, non-cementitious, highly resorbable and poorly-resorbable particulate substances, and further characterized by a specific particle size distribution that includes two or more different ranges of particle size. In an embodiment, the composition is Bond Apatite. In an embodiment, the device described herein includes any device disclosed in EP Patent No. 2252338, which is incorporated herein by reference in its entirety.

The compositions (e.g., adhesive compositions) described herein may include, in part or in whole, a composition known in the art, such as a composition described in any of the patents discussed above.

The compositions (e.g., adhesive compositions) described herein may be applied to surfaces that are dry, immersed in, submerged in, or damp with an aqueous medium (e.g., fresh water, saline, blood, sea water), or condensing water.

### EXAMPLES

### Example 1: Loading of an Exemplary Device with the Components of an Adhesive Composition

The dry components of the adhesive composition were loaded into the first chamber of the main body of a two-chambered device by filling the first chamber to a predetermined mass. The weight of the loaded device was verified using a scale to ensure the mass of the adhesive composition is within specification. This loading can be a manual process or an automated process using filling equipment known in the art.

Using automatic placement equipment, the sachet was then inserted into the main body of the device to a predefined depth. Air can escape from a chamber of the device, such as the second chamber when attaching the sachet, but not the powder component. This was visually verified by observing the depth of placement of the back end of the sachet. Placement of the sachet closes and seals the first chamber containing the solid components of the adhesive composition, since the sachet has an integral barrier (i.e., thin wall, same material) built into the proximal wall of the sachet body.

The liquid component(s) of the adhesive composition was filled into the sachet to a predetermined mass to ensures a proper liquid to powder ratio. As in the case of the solid components, the loading of the liquid component(s) can be a manual process or an automated process using filling equipment known in the art. This loading is verified using a scale to ensure the mass of the liquid component is within specification. The plunger was placed using automatic equipment to a predetermined depth to close the second chamber within the main body of the device containing the liquid. Pressurized air can escape from the second chamber when attaching the plunger, but not the liquid component.

### Example 2: Packaging of an Exemplary Device

Proper labeling was placed on the flexible foil pouch and the completed, filled device (from Example 1) was inserted nozzle end first (facing the chevron seal) into the foil pouch. The open end of the pouch was then sealed using a bar sealer to ensure a complete seal. The foil pouch was placed into an individual box prior to gamma irradiation. The completed, packaged device then was subjected to gamma irradiation (15-25 kGy) to sterilize the device.

### Example 3: Activation of an Exemplary Device within the Package

The packaged device (from Example 2) was activated by depressing the plunger until it was coincident with the device body. This alignment can be visually verified by observing the device through a transparent panel in the sealed package. The package was a laminate on both sides with a transparent panel on one side and an opaque aluminum panel on the other side. The package was sealed and had a low moisture vapor transmission rate through its materials and the seal.

Using finger pressure, the fork assembly of an agitator was opened to allow placement of the device. Placement can be performed by pressing on a fork to open a gap that will accommodate the device while it remains in the package. See, for example, FIG. 2.

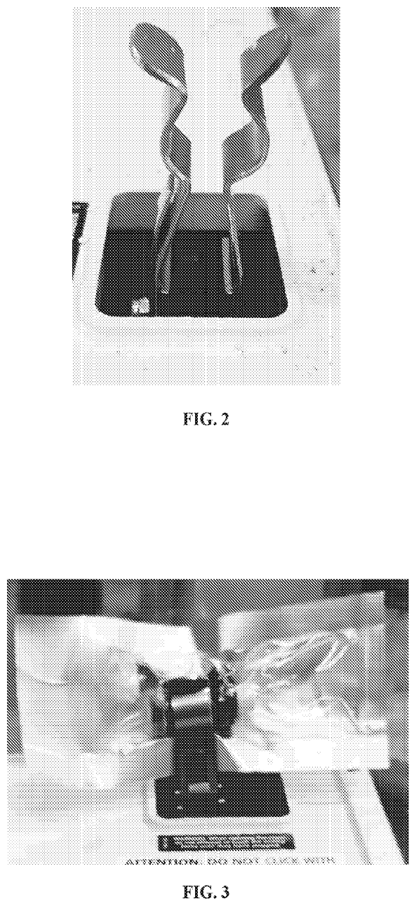

The activated packaged device was placed into an agitator that is located in a non-sterile surgical field by sliding it transverse to the longitudinal axis of the capsule body until the body of the device was captured around its midsection while it remains in its pouch. See, for example, FIG. 3.

The agitator was then run at 4500 Hz for 12+/−6 seconds to mix the components of the adhesive composition while the device was contained within the package.

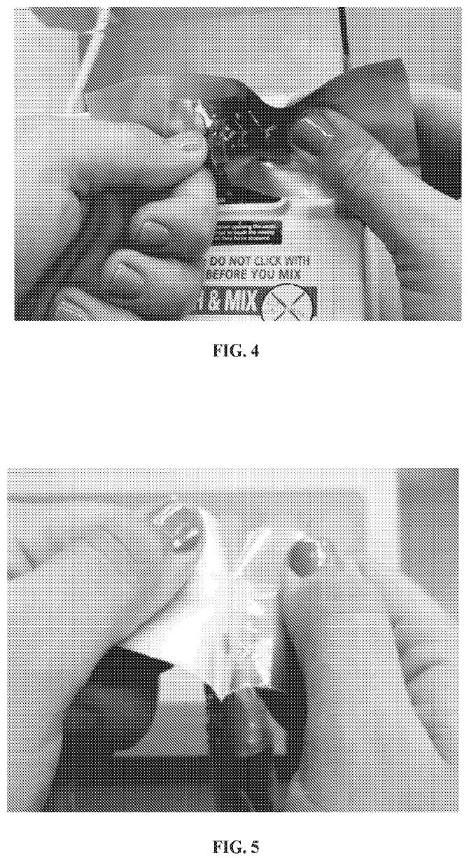

Upon agitator cycle completion, the fork assembly was released by gentle finger pressure to the fork assembly. See, for example, FIG. 4.

The package was then removed by sliding (e.g., superiorly) until the packaged device was free of the compressive force applied by the fork assembly.

The package was then opened by peeling a standard chevron seal until the device, with its mixed contents, was released onto a sterile surface using aseptic technique. Note, it is suggested that although the operator (e.g., circulating nurse, surgical assistant, or scrub technician) is working in a non-sterile surgical field that he/she wear sterile gloves when opening the package and transferring the device to the sterile field. See, for example, FIG. 5.

The device was then loaded into a sterile applicator device that is hand-actuated by a user in the sterile field to advance the plunger through the device in order to dispense the adhesive composition through the nozzle (e., tapered, straight, or curved) to the intended or target site. See, for example, FIG. 6.

### Example 4: Activation of an Exemplary Composition Stored within the Device

The packaged device as outlined in Example 2 may comprise any composition, e.g., an adhesive composition. Exemplary compositions may be Hydroset, NoriaN, Tetranite or other composition, e.g., as described herein. The device comprising the composition is loaded with components of each composition in the first and/or second chamber, packaged as described, and then sterilized. The components of a set of exemplary compositions contained within the device was then activated by depressing the plunger until it was coincident with the device body. This alignment was visually verified by observing the device through a transparent panel in the sealed package.

The activated packaged device was placed into an agitator is located in a non-sterile surgical field by sliding it transverse to the longitudinal axis of the capsule body until the body of the device was captured around its midsection while it remains in its pouch. See, for example, FIGS. 10A-B.

The agitator was then run at 4500 Hz for 12+/−6 seconds to mix the components of the composition while the device was contained within the package. Upon agitator cycle completion, the fork assembly was released by gentle finger pressure to the fork assembly. The package was then removed by sliding (e.g., superiorly) until the packaged device was free of the compressive force applied by the fork assembly.

The package was then opened by peeling a standard chevron seal until the device, with its mixed contents, was released onto a sterile surface using aseptic technique. Note, it is suggested that although the operator (e.g., circulating nurse, surgical assistant, or scrub technician) is working in a non-sterile surgical field that he/she wear sterile gloves when opening the package and transferring the device to the sterile field. See, for example, FIG. 9.

The device was then loaded into a sterile applicator device that is hand-actuated by a user in the sterile field to advance the plunger through the device in order to dispense the adhesive composition through the nozzle (e., tapered, straight, or curved) to the intended or target site. See, for example, FIGS. 8A-B.

1. A package comprising a device for storing, mixing, and administering a composition (e.g., adhesive composition), wherein
(a) the device comprises:
(i) a chamber within a main body;
(ii) a barrier;
(iii) a plunger;
(iv) a nozzle; and
(v) the components of the composition; and
(b) the package limits ingress of a contaminating element.

2. The package of claim 1, wherein the package comprises an interior environment for the device.

3. The package of claim 1, wherein the package comprises a clear or translucent region on at least one side of the package.

4. The package of claim 3, wherein the clear or translucent region comprises an entire aspect or side of the package.

5. The package of claim 1, wherein the package comprises at least two sheets or layers of material (e.g., a first sheet or layer of material and a second sheet or layer of material).

6. The package of claim 5, wherein the at least two sheets or layers of material are flexible.

7. The package of claim 5, wherein the at least two sheets or layers of material are rigid.

8. The package of claim 5, wherein the at least two sheets or layers of material are sealed around the device.

9. The package of claim 5, wherein the at least two sheets or layers of material are formed to hold a shape around the device.

10. The package of claim 5, wherein at least one sheet or layer of the material comprises a metal or polymer.

11. The package of claim 10, wherein the polymer comprises a polyolefin, polyamide, polyester, polyaryl, polycarbonate, or a mixture or co-extrusion thereof.

12. The package of claim 10, wherein the polymer is selected from polyvinyl chloride, polyvinylidene chloride, polyethylene, polyethylene terephthalate, polyethylene naphthalate, polyethylene terephthalate (e.g., biaxially-oriented polyethylene terephthalate), polystyrene, ethylene vinyl alcohol, or a mixture or co-extrusion thereof

13. The package of claim 10, wherein the metal is selected from aluminum or tin.

14. The package of claim 5, wherein the at least two sheets or layers of materials comprise a foil or laminate.

15. The package of claim 5, wherein the two sheets of material are sealed around the device using heat (e.g., a heat bar seal technique).

16. The package of claim 1, wherein the package is puncture resistant, abrasion resistant and/or resistant to tearing.

17. The package of claim 1, wherein the package comprises an opening mechanism (e.g., a chevron seal).

18. The package of claim 1, wherein the package comprises a grasping mechanism for mixing the device.

19. The package of claim 1, wherein the package provides an uncontaminated environment for the device.

20. The package of claim 1, wherein the package provides a sterile environment for the device.

21. The package of claim 1, wherein the package prevents egress of a contaminating element.

22. The package of claim 21, wherein the contaminating element comprises moisture, light, oxygen, a bacterium, a fungal cell, a spore, endotoxin, a pyrogen, or dust.

23. The package of claim 1, wherein the package further comprises a label.

24. The package of claim 1, wherein the package (e.g., including the device) is capable of undergoing sterilization (e.g. gamma irradiation).

25. The package of claim 1, wherein the device comprises more than one chamber.

26. The package of claim 1, wherein the device comprises a first chamber and a second chamber.

27. The package of claim 1, wherein the device comprises the components of a composition (e.g., an adhesive composition).

28. The package of claim 26, wherein one of the first chamber or the second chamber comprise the components of a composition.

29. The package of claim 26, wherein both of the first chamber and the second chamber comprise the components of a composition

30. The package of claim 1, wherein the composition comprises a multivalent metal compound and an acidic compound.

31. The package of claim 30, wherein the multivalent metal compound and the acidic compound are provided as dry components (e.g., a powder or granules) in a chamber of the device.

32. The package of claim 26, wherein a multivalent metal compound and the acidic compound are provided within the first chamber of the device.

33. The package of claim 26, wherein the second chamber of the device comprises an aqueous medium.

34. The package of claim 33, wherein the aqueous medium is water.

35. The package of claim 33, wherein the aqueous medium is a suspension of the compound selected in liquid suspension in the aqueous medium.

36. The package of claim 1, wherein the barrier separates a first chamber from a second chamber.

37. The package of claim 36, wherein the barrier comprises a material selected from a polymer or a metal.

38. The package of claim 36, wherein the barrier comprises a foil.

39. The package of claim 36, wherein the barrier has a diameter/thickness of less than 0.05 mm, e.g., 0.01 mm.

40. The package of claim 1, wherein the plunger comprises a shaft and a terminal end suitable for piercing a membrane.

41. The package of claim 40, wherein the terminal end of the plunger is in the shape of a cone or dome.

42. The package of claim 40, wherein the terminal end of the plunger is flat.

43. The package of claim 1, wherein the barrier is breached by the plunger when pressure is applied from a user.

44. The package of claim 1, wherein the plunger is depressed to breach the barrier while the device is still within the package.

45. The package of claim 1, wherein the barrier is breached by the terminal end of the plunger when pressure is applied, e.g., from a user.

46. The package of claim 43, wherein upon being breached, the contents within the main body of the device come into contact.

47. The package of claim 43, wherein the contents within the main body of the device are combined to form an implantable composition.

48. The package of claim 31, wherein upon being breached, the contents of the first chamber and the contents of the second chamber come into contact.

49. The package of claim 31, wherein the contents of the first chamber and the contents of the second chamber are combined to form a composition.

50. The package of claim 49, wherein the composition comprises an adhesive composition.

51. The package of claim 49, wherein the composition comprises a tissue repair composition.

52. The package of claim 1, wherein the package comprising the device is agitated to mix the components of a composition (e.g., an adhesive composition).

53. The package of claim 1, wherein the package comprising the device is placed into an agitator (e.g., a triturator) for agitation.

54. The package of claim 53, wherein the package comprising the device is subjected to between 5 and 30 seconds of mixing in an agitator (e.g., a triturator).

55. The package of claim 53, wherein the agitator (e.g., a triturator) comprises a device holder assembly (e.g., a fork assembly) comprising two arms.

56. The package of claim 55, wherein the device holder arms (e.g., fork arms) grasp the device within the package in such way that the barrier is not breached.

57. The package of claim 55, wherein the device holder arms (e.g., fork arms) clamp the device on the outer surface (e.g., the waist) of the device.

58. The package of claim 55, wherein the device holder arms (e.g., fork arms) are of a type which clamp the device but do not disturb the barrier or compromise sterility of the packaging.

59. The package of claim 1, wherein the nozzle of the device is less than 100 mm long (e.g., less than about 50 mm, 40 mm, 30 mm, 20 mm, 10 mm, 5 mm, 3 mm, 2 mm, 1 mm long).

60. The package of claim 1, wherein the nozzle comprises an inner diameter (e.g., at exit) of less than 5 mm (e.g., less than about 3 mm, 2 mm, 1 mm, 0.5 mm, or 0.25 mm).

61. The package of claim 1, wherein the barrier is a barrier separating a chamber in the device and the nozzle.

62. The package of claim 1, wherein the device further comprises a barrier separating the first chamber and the nozzle.

63. The package of claim 1, wherein the composition is dispensed from a chamber within the device (e.g., the first chamber and second chamber) into the nozzle by depression (translation, travel) of the plunger, e.g. driven by a force applied by a user.

64. The package of claim 1, wherein the device is removed from the package and inserted into an applicator to administer the adhesive composition.

65. The package of claim 1, wherein the composition is applied to an implantation site directly from the device, e.g., without removing the composition from the device and placing the composition into a second device for application.

66. The package of claim 30, wherein the multivalent metal comprises calcium (e.g., tetracalcium phosphate or tricalcium phosphate).

67. The package of claim 30, wherein the first chamber comprises between 10 mg and 2 g multivalent metal salt.

68. The package of claim 30, wherein the acidic compound comprises:
a compound of Formula (I) or a salt thereof: 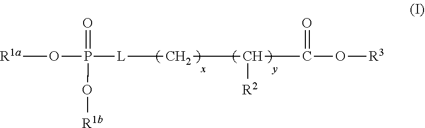
wherein
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4b} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3;
a compound of Formula (II) or a salt thereof: 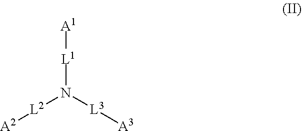
wherein:
each of A¹, A², and A³ is independently selected from an acidic group (e.g., a carboxyl or phosphonyl); and
and each of L¹, L², and L³ is independently bond, alkylene (e.g., C₁-C₆ alkylene), or heteroalkylene (e.g., C₁-C₆ heteroalkylene); or
a compound of Formula (III) or a salt thereof: 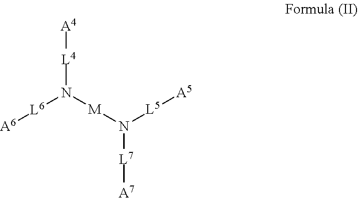
wherein:
each of A⁴, A⁵, A⁶, and A⁷ is independently an acidic group (e.g., a carboxyl or phosphonyl);
each of L⁴, L⁵, L⁶, and L⁷ is independently a bond, alkylene (e.g., C₁-C₆ alkylene), or heteroalkylene (e.g., C₁-C₆ heteroalkylene); and
M is alkylene (e.g., C₁-C₆ alkylene) or heteroalkylene (e.g., C₁-C₆ heteroalkylene).

69. The package of claim 68, wherein the acidic compound is a compound of Formula (I).

70. The package of claim 69, wherein the acidic compound is a compound of Formula (II) or (III).

71. The package of claim 69, wherein the acidic compound is phosphoserine.

72. The package of claim 26, wherein the first chamber comprises between 10 mg and 2 g acidic compound (e.g., a compound of Formula (I), e.g., phosphoserine).

73. The package of claim 26, wherein a chamber of the device comprises an additional component, e.g., an additive.

74. The package of claim 73, wherein the additional component comprises tricalcium phosphate (e.g., alpha tricalcium phosphate or beta tricalcium phosphate).

75. The package of claim 73, wherein the additional component comprises a polymer (e.g., PLGA).

76. The package of claim 73, wherein the additional component is tricalcium citrate.

77. The package of claim 73, wherein the additional component is a therapeutic agent.

78. The package of claim 1, wherein after mixing, the composition has a tacky consistency between 45 seconds to 4 minutes.

79. The package of claim 1, wherein after mixing, the composition has a hardened/cement state between 4 minutes and 20 minutes.

80. The package of claim 1, wherein after mixing, the composition has an adhesive state.

81. A method of preparing a composition (e.g., adhesive composition) comprising:
(a) disposing the components of a composition (e.g., adhesive composition) into a device comprising:
(i) a chamber within a main body;
(ii) a barrier;
(iii) a plunger; and
(iv) a nozzle;
(b) applying pressure/depressing the plunger of the device to breach the barrier, allowing the components of the adhesive composition to come into contact; and
(c) placing the device into an agitator to agitate the contents of the device; thereby preparing the composition (e.g., adhesive composition).

82. The method of claim 81, wherein the composition (e.g., adhesive composition) comprises a multivalent metal salt, a compound of Formulas (I), (II), (III), or (IV), and an aqueous medium.

83. The method of claim 81, wherein the device is enclosed within a package.

84. The method of claim 81, wherein the package is a package of any one of claims 1-78.

85. A method of preparing a composition (e.g., adhesive composition) comprising:
(a) disposing a multivalent metal compound and a compound (e.g., an acidic compound, a compound of Formula (I), (II), (III), or (IV)) into a device comprising:
(i) a first chamber and a second chamber within a main body;
(ii) a barrier separating the first chamber and the second chamber;
(iii) a plunger; and
(iv) a nozzle;
wherein each of the multivalent metal compound and the compound (e.g., an acidic compound, a compound of Formula (I), (II), (III), or (IV)) are disposed within the first chamber of the device, and an aqueous medium is disposed within the second chamber of the device;
(b) applying pressure/depressing the plunger of the device to compromise/break/displace or breach the barrier separating the first chamber and the second chamber, allowing the contents of the first chamber and the contents of the second chamber to come into contact
(c) placing the device into an agitating device to agitate the contents of the first chamber and second chamber; thereby preparing the composition (e.g., adhesive composition).

86. The method of claim 85, wherein the device is further disposed within a package that limits ingress of a contaminating element.

87. The method of claim 85, wherein after step (a), the device is disposed within a package that limits ingress and egress of a contaminating element.

88. A method of preparing a composition (e.g., adhesive composition) comprising:
(a) placing a package comprising a device into an agitator, wherein the device comprises:
(i) a first chamber comprising a multivalent metal compound and a compound (e.g., an acidic compound, a compound of Formula (I), (II), (III), or (IV));
(ii) a second chamber comprising an aqueous medium;
(iii) a plunger; and
(iv) a nozzle;
wherein the contents of the first chamber and the contents of the second chamber are exposed to one another; and
(b) allowing the agitator to agitate the contents of the device to form a composition (e.g., adhesive composition);
wherein the package limits ingress of a contaminating element, thereby preparing the composition (e.g., adhesive composition).

89. The method of claim 88, wherein the package is a package of any one of claims 1-80.

90. The method of claim 88, wherein the multivalent metal salt comprises calcium (e.g., tetracalcium phosphate) and the compound is a compound of Formula (I) (e.g., phosphoserine).

91. The method of claim 88, wherein the composition is used, e.g., directly or indirectly, in a sterile field (e.g., a surgical setting).

92. The method of claim 91, wherein the composition is applied to an implantation site directly from the device, e.g., without removing the composition from the device and placing the composition into a second device for application

93. The method of claim 91, wherein the composition is not transferred to a second device prior to use, e.g., in a sterile field (e.g., a surgical setting).

94. The method of claim 91, wherein the device is used in a manner to limits ingress of a contaminating element.
